(19) United States
(12) Patent Application Publication
(10) Pub. No.: US 2017/0027973 A1
(43) Pub. Date: Feb. 2, 2017

(54) MODULARION OF INVARIANT NATURAL KILLER T CELLS IN THE TREATMENT OF SEPSIS

(71) Applicant: LONDON HEALTH SCIENCES CENTRE RESEARCH INC., London (CA)

(72) Inventors: Ram Venkatesh ANANTHA, London (CA); John K. MCCORMICK, London (CA); S.M. Mansour HAERYFAR, London (CA)

(21) Appl. No.: 15/303,466

(22) PCT Filed: Apr. 10, 2015
(86) PCT No.: PCT/CA2015/050299

§ 371 Date: Oct. 11, 2016

Related U.S. Application Data
(60) Provisional application No. 61/978,467, filed on Apr. 11, 2014.

(51) Int. Cl. A61K 31/7028; A61K 35/17; G01N 33/50

(52) U.S. Cl. CPC A61K 31/7028; G01N 33/5091; A61K 35/17; G01N 2800/26

(57) ABSTRACT

The present disclosure is directed to the modulation of in variant Natural Killer T Cells (iNKT Cells) using a Th2-polarizing iNKT Cell agonist, such as an α-galactosylceramide, synthetic glycolipid OCH, or C20:2 α-galactosylceramide analog. Said Th2-polarizing iNKT Cell agonists are used the treatment of sepsis and/or apoptosis in a subject. A method of diagnosing early stage sepsis in a subject by measuring a ratio of circulating iNKT cells relative to total circulating T cells in a subject is also disclosed.

### FIELD OF THE INVENTION

The present invention relates to modulation of invariant natural killer T cells in the treatment of sepsis.

### BACKGROUND OF THE INVENTION

Sepsis is an overwhelming systemic inflammatory response to infection (1) that remains the leading cause of death among patients in intensive care units (2, 3), with a mortality rate approaching 35% (4). Despite a dramatic increase in the incidence of sepsis over the past thirty years (2), there are no selective therapeutic agents available to reduce the morbidity and mortality of this illness (4, 5).

There has been increasing interest in the role of invariant natural killer T (iNKT) cells in regulating host cytokine responses (6-10) and bridging the innate and adaptive immune arms of immunity during sepsis (11-13). iNKT cells are an evolutionarily conserved subset of T cells that are characterized by the expression of an invariant TCRα chain (Vα24-Jα18 in humans and Vα14-Jα18 in mice), and reactivity to self- and microbial-derived glycolipids presented by the monomorphic MHC class I-like molecule CD1d (9, 14). Once activated, iNKT cells secrete pro- and/or anti-inflammatory cytokines that can subsequently shape the course and nature of immune responses in a variety of disease states (9, 10, 14).

Although a number of studies have established iNKT cells as the principal initiators of an excessive pro-inflammatory response that promotes lethality in animal models of sepsis (15-17), little is known about the role of these cells in the context of human sepsis.

### SUMMARY OF THE INVENTION

The present invention relates to modulation of iNKT cells in the treatment of sepsis or to reduce apoptosis, and to the diagnosis of sepsis.

In one embodiment, the present invention provides for a method of treating sepsis in a subject. In one embodiment, the method includes administering to the subject a Th2-polarizing iNKT cell agonist.

In one embodiment of the method of treating sepsis in a subject, the Th2-polarizing iNKT cell agonist is a glycolipid ligand of iNKT cells.

In another embodiment of the method of treating sepsis in a subject, the Th2-polarizing iNKT cell agonist is α-Galactosyl Ceramide or an α-Galactosyl Ceramide analog or derivative.

In another embodiment of the method of treating sepsis in a subject, the Th2-polarizing iNKT cell agonist is synthetic glycolipid OCH.

In another embodiment of the method of treating sepsis in a subject, the Th2-polarizing iNKT cell agonist is C20:2.

In another embodiment, the present invention provides for a method of treating sepsis in a subject including administering to the subject a Th2 phenotype modulated iNKT cell.

In one embodiment of any of the previous methods of treating sepsis in a subject, sepsis is intra-abdominal sepsis.

In another embodiment, the present invention relates to the use of Th2-polarizing iNKT cell agonist for the treatment of sepsis, or Th2-polarizing iNKT cell agonist for use in the treatment of sepsis.

According to one embodiment of the use of Th2-polarizing iNKT cell agonist, the Th2-polarizing iNKT cell agonist is a glycolipid ligand of iNKT cells.

According to another embodiment of the use of Th2-polarizing iNKT cell agonist, the Th2-polarizing iNKT cell agonist is α-Galactosyl Ceramide or an α-Galactosyl Ceramide analog or derivative.

According to another embodiment of the use of Th2-polarizing iNKT cell agonist, the Th2-polarizing iNKT cell agonist is synthetic glycolipid OCH.

According to another embodiment of the use of Th2-polarizing iNKT cell agonist, the Th2-polarizing iNKT cell agonist is C20:2.

In another embodiment, the present invention relates to the use of a Th2 phenotype modulated iNKT cell in the treatment of sepsis in a subject.

According to one embodiment, the use of Th2-polarizing iNKT cell agonist or the use of the Th2 phenotype modulated iNKT cell of any of the previous embodiments, sepsis is intra-abdominal sepsis.

In another embodiment, the present invention relates to a method of reducing sepsis-induced apoptosis in a subject comprising administering to the subject a Th2-polarizing iNKT cell agonist.

In one embodiment of the method of reducing sepsis-induced apoptosis in a subject, the Th2-polarizing iNKT cell agonist is a glycolipid ligand of iNKT cells.

In another embodiment of the method of reducing sepsis-induced apoptosis in a subject, the Th2-polarizing iNKT cell agonist is α-Galactosyl Ceramide or an α-Galactosyl Ceramide analog or derivative.

In another embodiment of the method of reducing sepsis-induced apoptosis in a subject, the Th2-polarizing iNKT cell agonist is synthetic glycolipid OCH.

In another embodiment of the method of reducing sepsis-induced apoptosis in a subject, the Th2-polarizing iNKT cell agonist is C20:2.

The present invention, in another embodiment, provides for a method of reducing sepsis-induced apoptosis in a subject comprising administering to the subject a Th2 phenotype modulated iNKT cell.

The present invention, in another embodiment, provides for a Th2-polarizing iNKT cell agonist for use in reducing sepsis-induced apoptosis in a subject.

In one embodiment of the use of a Th2-polarizing iNKT cell agonist in reducing sepsis-induced apoptosis in a subject, the Th2-polarizing iNKT cell agonist is a glycolipid ligand of iNKT cells.

In another embodiment of the use of a Th2-polarizing iNKT cell agonist in reducing sepsis-induced apoptosis in a subject, the Th2-polarizing iNKT cell agonist is α-Galactosyl Ceramide or an α-Galactosyl Ceramide analog or derivative.

In another embodiment of the use of a Th2-polarizing iNKT cell agonist in reducing sepsis-induced apoptosis in a subject, the Th2-polarizing iNKT cell agonist is synthetic glycolipid OCH.

In another embodiment of the use of a Th2-polarizing iNKT cell agonist in reducing sepsis-induced apoptosis in a subject, the Th2-polarizing iNKT cell agonist is C20:2.

In another embodiment, the present invention relates to the use of a Th2 phenotype modulated iNKT cell for reducing sepsis-induced apoptosis in a subject.

In another embodiment, the present invention relates to a method of detecting early stage sepsis in a subject in need thereof, the method comprising: (a) measuring a test ratio of circulating iNKT cells relative to the total circulating T cells in the subject, and (b) comparing the test ratio to a control ratio of subjects without sepsis, wherein a test ratio higher than the control ratio is indicative of early stage sepsis.

In another embodiment, the present invention relates to a method of reducing apoptosis in a subject comprising administering to the subject a Th2-polarizing iNKT cell agonist.

In one embodiment of the method of reducing apoptosis of the present invention, the Th2-polarizing iNKT cell agonist is a glycolipid ligand of iNKT cells.

In another embodiment of the method of reducing apoptosis of the present invention, the Th2-polarizing iNKT cell agonist is α-Galactosyl Ceramide or an α-Galactosyl Ceramide analog or derivative.

In another embodiment of the method of reducing apoptosis of the present invention, the Th2-polarizing iNKT cell agonist is synthetic glycolipid OCH.

In another embodiment of the method of reducing apoptosis of the present invention, the Th2-polarizing iNKT cell agonist is C20:2.

In another embodiment, the present invention provides for a method of reducing apoptosis in a subject comprising administering to the subject a Th2 phenotype modulated iNKT cell.

In another embodiment, the present invention provides for a use of a Th2-polarizing iNKT cell agonist for reducing apoptosis in a subject.

In one embodiment of the use of Th2-polarizing iNKT cell agonist for reducing apoptosis in a subject, the Th2-polarizing iNKT cell agonist is a glycolipid ligand of iNKT cells.

In another embodiment of the use of Th2-polarizing iNKT cell agonist for reducing apoptosis in a subject, the Th2-polarizing iNKT cell agonist is α-Galactosyl Ceramide or an α-Galactosyl Ceramide analog or derivative.

In another embodiment of the use of Th2-polarizing iNKT cell agonist for reducing apoptosis in a subject, the Th2-polarizing iNKT cell agonist is synthetic glycolipid OCH.

In another embodiment of the use of Th2-polarizing iNKT cell agonist for reducing apoptosis in a subject, the Th2-polarizing iNKT cell agonist is C20:2.

The present invention, in another embodiment, provides for a use of a Th2 phenotype modulated iNKT cell for reducing apoptosis in a subject.

In one embodiment of the methods of reducing apoptosis (including sepsis-induced) of the present invention or uses for reducing apoptosis (including sepsis-induced), the methods or uses reduce apoptosis in splenic B and T lymphocytes and macrophages.

### BRIEF DESCRIPTION OF THE DRAWINGS

The following figures illustrate various aspects and preferred and alternative embodiments of the invention.

FIG. 1: Characterization of iNKT cell populations in the sera of critically-ill patients. (a) Representative flow cytometry plots of peripheral blood sampled from a septic and non-septic patient. (b) Histograms (median±SEM) comparing frequency of T cells, NK cells, and iNKT:T cell ratios in septic and non-septic patients in the intensive care unit. *p<0.05 by Mann Whitney U test (c) Kaplan-Meier survival curves from time of blood collection to time of discharge.

FIG. 2: iNKT cells are pathogenic in intra-abdominal sepsis (IAS). (a) B6 and iNKT-deficient Jα18−/− mice (n=6) were injected with fecal slurry (90 mg/mL) to induce IAS and monitored during the experimental timeline. Murine sepsis scores were significantly higher compared to sham-treated B6 and Jα18−/− mice (injected i.p. with normal saline [NS]) and Jα18−/− mice with IAS (n=6 for sham B6 and Jα18−/− mice each, n=10, n=6 for septic B6 and Jα18−/− mice respectively). ***p<0.001 by two-way ANOVA test. (b) Mortality for B6 mice with IAS were significantly higher than sham B6 and Jα18−/− mice, as well as septic Jα18−/− mice (n=6 for sham B6 and Jα18−/− mice each, n=10, n=6 for septic B6 and Jα18−/− mice respectively). ***p<0.001 by log-rank test.

FIG. 3: Tissue-specific distribution of iNKT cells is altered during IAS. (a) The distribution of T and iNKT cells in the spleen and omentum is altered significantly in IAS, but remains unchanged in the liver (n=7, n=10 in sham and IAS groups respectively). Percentages of cell populations are represented as means±SEMs. ***p<0.0001, **p<0.001, *p<0.05 by Mann Whitney U test (b) Quantitative RT-PCR using custom-designed probes designed for detecting the invariant TCR demonstrates a significantly elevated expression of TCR transcripts within the spleen, liver, and omentum following IAS. Relative fold changes of the expression of the invariant TCR were calculated based on the ΔΔCt method after normalizing to GAPDH as an internal control (n=3, n=5 in sham and IAS groups respectively). ***p<0.0001, **p<0.001, *p<0.05 by Mann Whitney U test.

FIG. 4: Th2-polarizing glycolipid OCH reduces disease severity in IAS. C57BL/6 (B6) mice were concomitantly injected i.p. with fecal slurry (FS; 500 μL of 90 mg/mL solution) to induce intra-abdominal sepsis (IAS), and vehicle, OCH, or KRN7000. (a) OCH-treated mice had significantly prolonged survival compared to vehicle- and KRN7000-treated mice (n=19, n=15, n=8 for OCH, vehicle, and KRN7000 groups respectively). ***p<0.001 by log-rank test (b) OCH-treated mice demonstrated significantly reduced disease severity compared to vehicle-treated and KRN7000-treated mice (n=19, n=15, and n=8 mice respectively for OCH, KRN7000, and vehicle groups). ***p<0.001 by two-way ANOVA with Bonferroni post-test. (c) iNKT-deficient Jα18−/− mice were given FS (500 μL of a 90 mg/mL solution) to induce IAS and concomitantly treated with OCH or vehicle. Murine sepsis scores were similar between vehicle and OCH-treated mice (n=3 per group). (d) Administration of OCH and KRN7000 resulted in significantly reduced detection of iNKT cells among septic B6 mice compared to vehicle treatments. The percentages of T cells remained unchanged with administration of iNKT-specific glycolipid agonists (n=6, n=4, n=6, and n=3 for vehicle, OCH, Vehicle (KRN7000) and KRN7000 groups respectively). *p<0.05, **p<0.01 by Mann-Whitney U test. (d) Bacterial counts in blood and multiple organs were similar between vehicle-, OCH-, and KRN7000-treated mice with sepsis (n=7-9 per group). Data are representative of at least three independent experiments.

FIG. 5: Cytokine levels in the sera and spleens of septic B6 mice. (a) Sera and spleen homogenates from vehicle-, OCH-, and KRN7000-treated B6 mice with intra-abdominal sepsis (IAS) were analyzed for 32 inflammatory cytokines by multiplex array, and displayed as a heat map (n=4 mice per group). Concentrations of iNKT cell-specific cytokines are shown from sera (b) and spleen homogenates (c) of septic mice treated with vehicle, OCH, or KRN7000 (n=4-8 per group). Concentrations of cytokines are shown in pg/mL. *p<0.05, **p<0.01, ***p<0.001 by one-way ANOVA with post-hoc Tukey's multiple comparison test. Data are representative of at least three independent experiments.

FIG. 6: Histopathology of septic B6 mice treated with glycolipid agonists of iNKT cells. (a) Treatment with OCH significantly reduced apoptosis within the spleen compared to vehicle- and KRN7000-treated mice with intra-abdominal sepsis (IAS), both by hematoxylin and eosin staining, as well as TUNEL staining. Lymphocyte migration to the omentum is also ameliorated in OCH-treated mice compared to vehicle- and KRN7000-treated mice. There were no histopathological differences in the liver. Images are representative of 4 animals per treatment group (size bar, 50 μm). (b) Histopathological scoring of the degree of apoptosis observed within the spleens of sham and septic B6 mice treated with vehicle, KRN7000, or OCH (n=4 animals per treatment group). Apoptosis was defined histologically by the presence of cell clusters with nuclear shrinkage (karyorrhexis), dark eosinophilic cytoplasm, intact plasma membrane, and relative paucity of surrounding inflammatory cells within the splenic follicles on H&E staining. Scores assigned to each animal by a blinded independent pathologist were as follows: 0 for complete absence of apoptosis; 1 for mild presence of apoptosis (0-15% per follicle); 2 for moderate apoptosis (16-30% per follicle); and 3 for severe apoptosis (31-45% per follicle). ***p<0.0001 by two-tailed Mann Whitney U test.

FIG. 7: Analysis of apoptotic cell populations in the spleens of septic B6 mice. Splenocytes from sham and septic B6 mice treated with OCH, KRN7000, or vehicle were stained for T, B, and Natural Killer (NK) cells, and macrophages, and further stained for Annexin V (a marker for early apoptosis) and 7-AAD viability dye. Early and late apoptotic cells (Annexin V+7AAD- and Annexin V+7AAD+ cells respectively) were quantified and compared between treatments. OCH treatment significantly reduced apoptosis among T and B cells, as well as macrophages, but not NK cells. (n=3-6 mice per group). *p<0.05, **p<0.01, ***p<0.001 by one-way ANOVA with post-hoc Tukey's multiple comparison test. Data are representative of 3 independent experiments.

FIG. 8: Gating strategy to identify the percentage of apoptotic and necrotic immune cell populations. CD3-APC is shown as an example, but the same strategy was used for macrophages (F480-APC), B cells (B220-APC), and Natural Killer (NK) cells (NK1.1-PE).

FIG. 9: Adoptive transfer of iNKT cells into iNKT-deficient Jα18−/− mice. iNKT cells were isolated and sorted from GFP-expressing transgenic mice, and injected i.v. into Jα18−/− mice. After 18 hours, mice were administered a fecal slurry (500 μL of a 90 mg/mL solution) intraperitoneally to induce IAS and monitored for 24 hours. (a) Adoptive transfer of iNKT cells significantly increased the severity of sepsis compared to Jα18−/− mice that did not receive iNKT cells. (b) Adoptively-transferred iNKT cells moved into the omentum of Jα18−/− mice following IAS, as detected by flow cytometry, compared to adoptively-transferred iNKT cells in sham Jα18−/− mice.

FIG. 10: Effect of glycolipid agonists on cytokine expression in naïve B6 mice, and on iNKT cells in septic B6 mice. (a) Naïve B6 mice were injected i.p. with 4 μg OCH or KRN7000 or C20:2, and bled at 2, 12, and 24 hours post-injection. Serum samples were assayed for IL-4 and IFN-γ by enzyme-linked immunosorbent assay (ELISA). Each data point shows mean (±SEM) of two or three mice from one representative experiment. Vehicle-treated mice had cytokine levels below limits of detection. (b) B6 mice were given an intraperitoneal injection of fecal slurry (500 μL of a 90 mg/mL solution) to induce IAS and concomitantly treated with 4 μg of vehicle, OCH, or KRN7000. After 24 hours, mice were sacrificed, and cell suspensions from the liver and spleen were stained for the flow cytometric detection of CD1d tetramer+ TCRβ+iNKT cells.

FIG. 11: C57BL/6J (B6) mice were injected intraperitoneally with 500 μL of FS (90 mg/mL) to induce IAS, and concomitantly injected with 4 μg of the glycolipid C20:2 or vehicle solution. (a) Murine Sepsis Scores for septic mice treated with C20:2 or vehicle (n=5, n=10 mice for C20:2 and vehicle groups respectively). ***p<0.001 by two-way ANOVA test. (b) After 24 hours, septic B6 mice treated with C20:2 were sacrificed, and the liver, spleen, and omentum were removed and processed for histopathological analysis. These images are representative of 5 septic B6 mice that were treated with C20:2 (size bar, 25 μm).

### DESCRIPTION OF THE INVENTION

### Definitions

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. In order to aid in the understanding and preparation of the within invention, the following illustrative, non-limiting, examples are provided.

A “subject” in the context of the present invention includes and encompasses mammals such as humans, primates and livestock animals (e.g. sheep, pigs, cattle, horses and donkeys), laboratory test animals such as mice, rabbits, rats and guinea pigs, and companion animals such as dogs and cats. It is preferred for the purposes of the present invention that the mammal is a human.

“Treatment” is used herein to refer to any regimen that can benefit a human or non-human mammal. The treatment may be in respect of an existing condition or may be prophylactic (preventative treatment). Treatment may include curative, alleviation or prophylactic effects.

As used herein, modulation of the iNKT cell response means the partial or complete down-regulation of proliferation and/or activity of the iNKT cells.

Administration

The Th2-polarizing iNKT cell agonist for use in the present invention may be administered alone but will preferably be administered as a pharmaceutical composition, which will generally comprise a suitable pharmaceutical excipient, diluent or carrier selected depending on the intended route of administration.

The Th2-polarizing iNKT cell agonist, including α-galactosyl ceramide or an analog or derivative of the α-galactosyl ceramide, such as OCH and C20:2 for use in the present invention may be administered to a patient in need of treatment via any suitable route. The precise dose will depend upon a number of factors, including the precise nature of the form of the iNKT cell agonist to be administered.

Examples of OCH glycolipids that may be used in the present invention, include those cited in U.S. Pat. No. 8,367,623, the content of which is incorporated by reference. Examples of C20:2 glycolipids that may be used in the present invention, include those cited in U.S. Pat. No. 8,022,043, the content of which is incorporated by reference.

Although the preferred route of administration is parenterally (including subcutaneous, intramuscular, intravenous), some further suitable routes of administration include (but are not limited to) oral, rectal, nasal, topical (including buccal and sublingual), infusion, vaginal, intradermal, intraperitoneally, intracranially, intrathecal and epidural administration or administration via oral or nasal inhalation, by means of, for example, a nebuliser or inhaler, or by an implant.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or Lactated Ringer's injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al., Biopolymers 22(1): 547-556, 1985), poly (2-hydroxyethyl-methacrylate) or ethylene vinyl acetate (Langer et al., J. Biomed. Mater. Res. 15: 167-277, 1981, and Langer, Chem. Tech. 12:98-105, 1982).

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins 20th edition (Dec. 15, 2000) ISBN 0-912734-04-3 and Pharmaceutical Dosage Forms and Drug Delivery Systems; Ansel, H. C. et al. 7th Edition ISBN 0-683305-72-7, the entire disclosures of which are herein incorporated by reference.

Pharmaceutical Compositions

As described above, the present invention extends to a pharmaceutical composition for the modulation of iNKT cells for use in the treatment of sepsis or to reduce sepsis-induced apoptosis, and in particular the down regulation of a iNKT cell response wherein the composition comprises at least a α-galactosyl ceramide or an analog or derivative of the α-galactosyl ceramide, such as OCG and C20:2.

Pharmaceutical compositions according to the present invention, and for use in accordance with the present invention, may comprise, in addition to active ingredient (i.e. a α-galactosyl ceramide or an analog or derivative of the α-galactosyl ceramide, such as OCG and C20:2), a pharmaceutically acceptable excipient, carrier, buffer stabiliser or other materials well known to those skilled in the art.

Such materials should be non-toxic and should not interfere with the efficacy of the active ingredient. The precise nature of the carrier or other material will depend on the route of administration, which may be, for example, oral, intravenous, intranasal or via oral or nasal inhalation.

The formulation may be a liquid, for example, a physiologic salt solution containing non-phosphate buffer at pH 6.8-7.6, or a lyophilised or freeze dried powder.

Dose

The composition is preferably administered to a subject in a therapeutically “effective amount” or a “desired amount”, this being sufficient to show benefit to the subject.

As defined herein, the term an “effective amount” means an amount necessary to at least partly obtain the desired response, or to delay the onset or inhibit progression or halt altogether the onset or progression of a particular condition being treated.

The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials.

Prescription of treatment, e.g. decisions on dosage etc, is ultimately within the responsibility and at the discretion of general practitioners, physicians or other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration. However, 0.001 mg of the a α-galactosyl ceramide or analog or derivative, to 5,000 mg/day/person is ordinarily suitable with 0.01 mg to 500 mg/day/person preferred and 0.5 mg to 100 mg/day/person more preferred.

A broad range of doses may be applicable. Dosage regimes may be adjusted to provide the optimum therapeutic response. For example, several divided doses may be administered daily, weekly, monthly or at other suitable time intervals or the dose may be proportionally reduced as indicated by the exigencies of the situation.

In order to aid in the understanding and preparation of the within invention, the following illustrative, non-limiting, examples are provided.

### Examples

### Materials and Methods

Patients and Clinical Samples

Approval of the study protocol for both the scientific and ethical aspects was obtained from the Western University Research Ethics Board for Health Sciences Research Involving Human Subjects. This study was conducted in accordance with the guidelines of the World Medical Association's Declaration of Helsinki (23).

Patients aged 18 years and older with a diagnosis of sepsis, severe sepsis or septic shock upon admission to the Medical-Surgical Intensive Care Unit (ICU) at London Health Sciences Centre (LHSC) University Hospital and the Critical Care and Trauma Centre at LHSC Victoria Hospital were prospectively recruited. The first day following ICU admission was considered day 1 in the analysis. Sepsis, severe sepsis, and septic shock were defined according to established guidelines (24). Severity of illness was assessed on the Acute Physiology and Chronic Health Evaluation II (APACHE II) score for the first 24 hours following diagnosis (25, 26). Exclusion criteria were the presence of immunodeficiency or concomitant immunosuppressive therapy, pregnancy, Do Not Resuscitate (DNR) status, and cardiac arrest. Informed consent was obtained directly from each patient or his or her legal representative before enrolment.

Standard cultures in biological samples guided by the presumptive source of the septic insult were performed to assess the presence of bacterial and fungal infection. Species identification was conducted by the LHSC Clinical Microbiology Laboratory. Potentially contaminant microorganisms were not considered. Blood was collected in heparinized vacuum tubes, and peripheral blood mononuclear cells (PBMCs) were isolated using Ficoll (Invitrogen) gradient centrifugation, according to published methods (27). PBMCs were subsequently stained for detection of iNKT cells by flow cytometry.

Animals

All animal experimentation was carried out in strict accordance with the recommendations and guidelines established by the Canadian Council on Animal Care as well as institutional regulations. Mouse studies were performed according to a protocol approved by the Western University Animal Care and Veterinary Services.

Female 10-12 week old mice were used for all experiments. All mice were maintained in a specific pathogen-free facility at Western University. C57BL/6 (B6) mice were obtained from Charles River Canada Inc. (St Constant, Quebec, Canada). Jα18−/− mice, which are on a B6 background and lack iNKT cells (28), were obtained from Dr. Luc Van Kaer (Vanderbilt University, Nashville, Tenn., USA). GFP-expressing transgenic mice are B6 background mice with omnipresent enhanced GFP expression under the β-actin promoter, and were kindly provided by Dr. Stephen Kerfoot (Western University, London, Ontario).

Murine Intra-Abdominal Sepsis (IAS) Model

Stool from the cecum of euthanized naïve B6 mice was expressed through an enterotomy, homogenized, and suspended in 0.9% normal saline (NS) to produce a fecal slurry at a final concentration of 90 mg/mL, for use in experiments on the same day. Mice were injected intraperitoneally with 500 μL fecal slurry or 0.9% normal saline (sham group). The mice were monitored and scored by two independent investigators—one of whom was blinded to the treatment—every 2-3 hours for the first 12 hours, and then every 1-2 hours thereafter. A murine sepsis score (MSS) was assigned to each mouse based on activity, eye movement, response to exogenous stimuli, breathing rate and pattern, and degree of piloerection (Shrum et al. BMC Research Notes 2014, 7:233). The MSS is a reliable and robust scoring system that has excellent inter-rater reliability (intra-class coefficient=0.96), high internal consistency (Cronbach α=0.92), and correlates with levels of certain pro-inflammatory cytokines such as IFN-γ and IL-1β during the experimental timeline (Shrum et al. BMC Research Notes 2014, 7:233). Mice were euthanized at 24 hours, or earlier if they were deemed to be in significant distress based on our scoring model. Euthanasia was carried out as described above. At the time of sacrifice, intra-cardiac blood was drawn into 1.5 mL micro-centrifuge tubes (BD Biosciences) and centrifuged for serum at 1000×g for 30 minutes at 4° C.

Glycolipids

Lyophilized OCH was generously provided by the National Institutes of Health (NIH) Tetramer Core Facility (Emory University, Atlanta, Ga.). Each vial containing 0.2 mg of OCH was solubilized in a vehicle solution containing 1 mL of sterile distilled water, 0.5% Tween 20, 56 mg of sucrose and 7.5 mg of histidine, and stored as aliquots at 4° C. until use. KRN7000 (α-GalCer, C26:0/C18:0) was purchased from Funakoshi Co. Ltd. (Tokyo, Japan), solubilized at 1 mg/ml in dimethylsulphoxide (DMSO), and stored as aliquots at −20° C. until use (29); the control vehicle was 2% DMSO in phosphate-buffered saline (PBS). C20:2 was synthesized and used as previously published (30, 31). For in vivo experiments, mice were injected intraperitoneally (i.p.) with a single dose of glycolipid (4 μg/dose) (30) within twenty minutes after induction of IAS.

Antibodies

Mouse Studies. APC-conjugated PBS-57-loaded and -unloaded CD1d tetramers for staining mouse iNKT cells were kindly provided by the NIH Tetramer Core Facility (30). FITC-conjugated anti-TCR-β, APC-conjugated F4/80, APC-conjugated B220, and PE-conjugated NK1.1 were purchased from eBiosciences or BD Biosciences.

Human Studies. APC-conjugated PBS-57-loaded and -unloaded CD1d tetramers for staining human iNKT cells were kindly provided by the NIH Tetramer Core Facility while FITC-conjugated anti-CD3 (SK7), and PE-conjugated anti-CD56(B159) were purchased from BD Biosciences.

Determination of Microbial Growth from Tissue Homogenization and Peripheral Blood

Whole hearts, lungs (left and right), kidneys (left and right), spleen, and liver were removed from euthanized mice and homogenized in 5 mL of PBS. Homogenates were serially diluted 1:10 in PBS and plated on bovine heart infusion (BHI) agar. Plates were grown aerobically at 37° C. overnight to determine tissue colony-forming units (CFU). Intra-cardiac blood (10 μL) was collected in a heparinized syringe from the right ventricle, serially diluted 1:10 with PBS, and plated on BHIagar to determine blood CFU.

Preparation of Murine Hepatic, Splenic, and Omental Cell Suspensions

To obtain hepatic lymphoid mononuclear cells (MNCs), mice were euthanized, and livers were flushed with sterile PBS before they were harvested and pressed through a 40-μm nylon mesh. The resulting homogenate was washed in cold PBS, resuspended in a 33.75% Percoll PLUS solution (GE Healthcare Bio-Sciences) and spun at 700×g for 12 min at room temperature. The pelleted cells were then treated with ACK lysis buffer to remove erythrocytes and washed in cold PBS prior to staining. To obtain omental lymphoid MNCs, the spleen, pancreas, and omentum were removed en-bloc and suspended in ice-cold PBS. The omenta floated above the spleen-pancreas complex and were removed and processed similar to the liver. Spleens were processed with a tissue homogenizer, and the resulting homogenate was washed in cold PBS. The pelleted cells were treated with ACK lysis buffer for 4 minutes to remove erythrocytes, and washed in cold PBS prior to staining.

Adoptive Transfer of iNKT Cells into Jα18−/− Mice

Hepatocytes and splenocytes were isolated as previously described from transgenic GFP mice. CD4+ T cell populations were obtained using EasySep® Mouse CD4+ T cell enrichment kit (Stem Cell Technologies) as per the manufacturer's instructions. iNKT cells were further enriched by sorting with anti-CD3 and anti-CD1d tetramer on a FACSArialII flow cytometric cell sorter (London Regional Flow Cytometry Facility, London, Ontario). Cell populations were used only when purity was >95% as determined by flow cytometry. For the adoptive transfer experiments, 5×105 iNKT cells were transferred i.v. into Jα18−/− mice. Eighteen hours after the transfer, mice were given IAS and monitored as already described.

Flow Cytometry

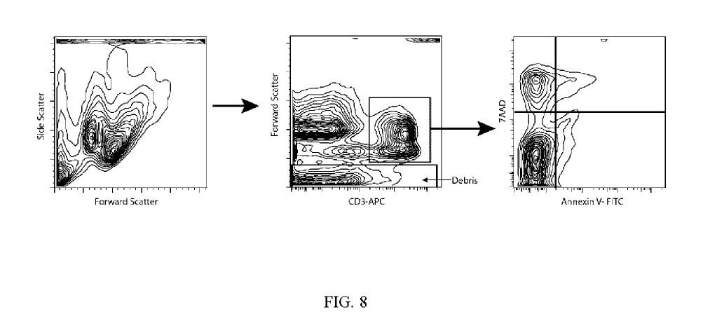

Mouse hepatic, splenic, and omental cells (1×106), and human PBMCs (1×106) were washed with cold FACS buffer [PBS+2% fetal bovine serum (FBS)+0.1% sodium azide] and incubated with 5 μg/ml anti-mouse CD16/CD32 mAb (clone 2•4G2, Fc-block, eBiosciences) for 20 min on ice before staining with fluorescent monoclonal antibodies (mAbs) diluted in FACS buffer at 4° C. for 30-40 min. Cells were then washed and flow cytometry was performed using FACSCanto II (BD) with FlowJo software (Treestar). The gating strategy used for the analysis of apoptotic and necrotic cells is shown in FIG. 8.

Quantitative Real-Time PCR

Total RNA was isolated from hepatic, splenic, and omental tissues using the TRIzol reagent (Invitrogen) and resuspended in nuclease-free water (Invitrogen). Quality control of samples was carried out using a Nanodrop ND-1000 spectrophotometer. cDNA was prepared using 500 ng of RNA by Superscript III RNase H-Reverse Transcriptase with oligodT priming (Invitrogen). Quantitative real-time PCR reactions were carried out in triplicate from every transcription reaction using the ABI Prism 7900HT apparatus (Perkin Elmer) with Taqman probes (Invitrogen). The sequences of the primers and Taqman probes used in this study were as follows: Vα14: 5′-TGGGAGATACTCAGCAACTCTGG-3′; Jα18: 5′-CAGGTATGACAATCAGCTGAGTCC-3′; and Vα14 probe FAM: 5′-FAM-CACCCTGCTGGATGACACTGCCAC-TAMRA-3′. Quantitative analysis was performed by the comparative ΔΔCt method by using the Taqman GAPDH Gene Expression Assay (Invitrogen) as an internal control. Minimum information for Publication of Quantitative Real-time PCR Experiments (MIQE) guidelines was followed (32).

ELISA Assays

Cytokine concentrations were determined by commercially available specific ELISA assays for IL-4 and IFN-γ (Ready-Set-Go kits, eBioscience). ODs were measured on a Benchmark Microplate Reader (Bio-Rad) at a wavelength of 450 nm for both cytokines.

Multiplex Cytokine Quantification Assay

Serum was analyzed by bead-based multiplex assay for 32 different cytokines, chemokines, and growth factors (Eve Technologies, Calgary, Alberta, Canada) including granulocyte-colony stimulating factor (G-CSF), granulocyte macrophage-colony stimulating factor (GM-CSF), interferon-gamma (IFN-γ), interleukin (IL)-1α, IL-1β, IL-2, IL-3, IL-4, IL-5, IL-6, IL-7, IL-9, IL-10, IL-12 (p40), IL-12 (p70), IL-13, IL-15, IL-17A, IP-10, keratinocyte chemo-attractant (KC), leukemia inhibitory factor (LIF), lipopolysaccharide-induced CXC chemokine (LIX), monocyte chemotactic protein (MCP)-1, monocyte-colony stimulating factor (M-CSF), monokine induced by gamma interferon (MIG), macrophage inflammatory protein (MIP)-1α, MIP-1β, MIP-2, Regulated on Activation, Normal T cell Expressed and Secreted (RANTES), tumour necrosis factor (TNF)-α, and vascular endothelial growth factor (VEGF). Multiplex data was visualized using a cytokine heat map that was generated using Matrix2png (33).

Histology Analysis and TUNEL Staining

Liver, spleen, and omentum from sham B6 mice, and vehicle-, OCH-, and KRN7000-treated septic B6 mice were fixed in 10% neutral buffered formalin, embedded in paraffin, sectioned, and stained with H&E or subjected to TUNEL staining using a commercially available kit (Calbiochem). At least four stained sections per spleen from four animals per treatment group were examined and scored for apoptosis by a board-certified veterinary pathologist (Welch) who was blinded to the study design. Apoptosis was defined histologically by the presence of cell clusters with nuclear shrinkage (karyorrhexis), dark eosinophilic cytoplasm, intact plasma membrane, and relative paucity of surrounding inflammatory cells within the splenic follicles on H&E staining (34). Scores assigned to each animal were as follows: 0 for complete absence of apoptosis; 1 for mild presence of apoptosis (0-15% per follicle); 2 for moderate apoptosis (16-30% per follicle); and 3 for severe apoptosis (31-45% per follicle). Photomicrographs were taken using a Nikon DS-Fi1 digital camera using NIS-elements software.

Statistical Analysis

For murine experiments, statistical comparisons were performed using analysis of variance (ANOVA) or Mann Whitney U test (GraphPad Prism). Survival curves were calculated by the Kaplan-Meier method. For cytokine analysis, results from multiple experiments were pooled and analyzed by one-way ANOVA with post-hoc comparisons using Tukey's tests.

For human subjects, differences between groups were assessed using the Mann-Whitney U-test or Chi-square test for continuous and categorical variables respectively. Survival curves were calculated by the Kaplan-Meier method and compared by log-rank test. In all analyses, p<0.05 was considered statistically significant.

Results

Peripheral Blood iNKT Cells are Elevated in Patients with Sepsis/Severe Sepsis

We first sought to determine if patients with sepsis had an altered frequency of iNKT cells in their peripheral blood compared to non-septic patients. We prospectively evaluated thirty patients who were admitted to LHSC Critical Care and Trauma Centre for sepsis or non-sepsis-related critical illness; 23 patients were diagnosed with sepsis/severe sepsis, while 7 patients were non-septic trauma patients (Table 1). In the non-septic group, 3 patients (43%) had sustained traumatic head injuries and 4 patients (57%) had emergency surgery for trauma (2 liver resections; 1 abdominal aortic surgery; 1 spine stabilization operation). Groups were similar in age and severity of illness, as calculated by the APACHEII score (25). However, the gender distribution was significantly different between the two groups, with a preponderance of males in the non-septic group (p<0.0001). Most of the patients in the septic group had intra-abdominal sepsis (44%) or lower respiratory tract infections (39%) as confirmed by diagnostic tests. In 30% and 17% of septic patients respectively, a single Gram-positive or Gram-negative pathogen was identified, while multiple organisms were identified in 30% of the septic group. In 17% of septic patients, the microbial agent was not identified, while 1 patient (4%) had fungal candidemia (Table 1).

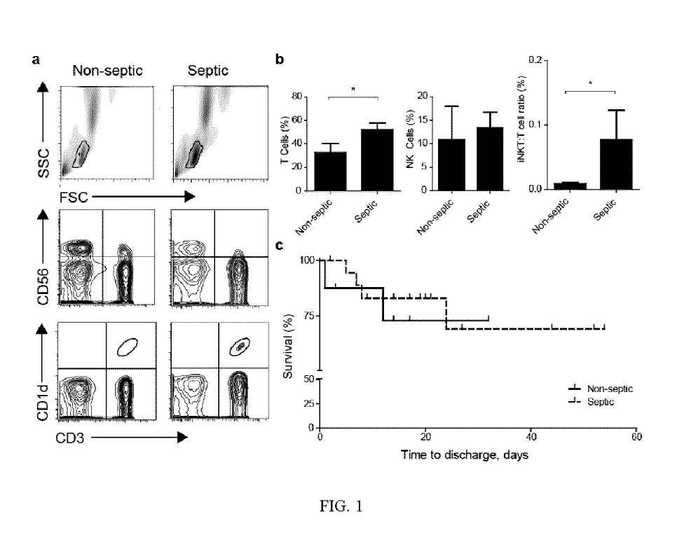

When lymphocyte subpopulations were assessed by flow cytometry and compared, the septic group had a higher median percentage of T cells among total lymphocytes (Table 2; FIGS. 1a and 1b). Moreover, the iNKT:T cell ratio was significantly higher in the septic group (Table 2). Patients in the septic group stayed in hospital for a significantly longer time, although in-hospital mortality was similar between the two groups (FIG. 1c).

iNKT Cells are Pathogenic in Intra-Abdominal Sepsis

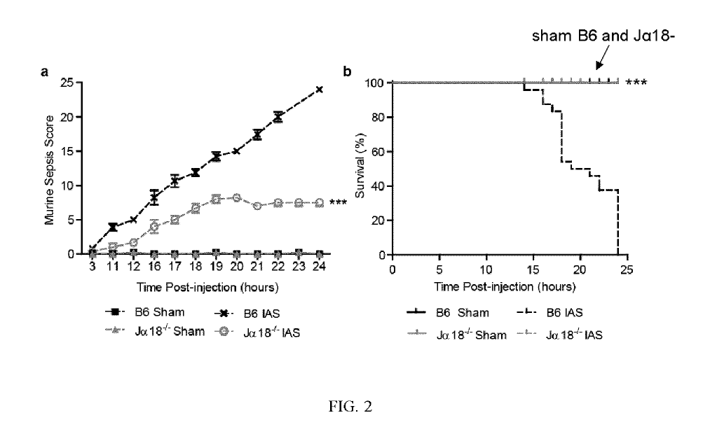

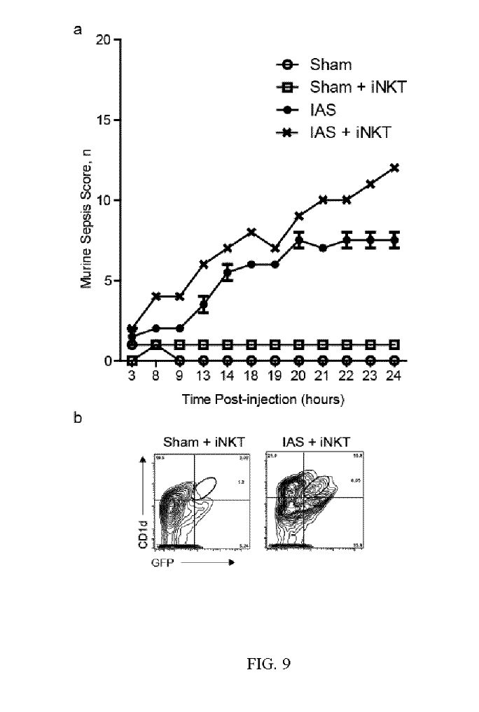

Given the finding of elevated iNKT cell proportions in human sepsis/severe sepsis, and the studies that have demonstrated the pathogenicity of iNKT cells in animal models mimicking chronic polymicrobial sepsis (11, 18), we studied iNKT cells in a well-controlled mouse model of acute intra-abdominal sepsis (IAS; Shrum et al. BMC Research Notes 2014, 7:233). Since iNKT cells can rapidly produce pro- and/or anti-inflammatory cytokines in response to stimuli and shape the subsequent immune responses in various diseases (35, 36), we hypothesized that these cells would affect disease severity and survival in IAS. Compared to C57BLJ6 (B6) mice, we observed a significant reduction in sepsis severity (FIG. 2a) and mortality (FIG. 2b) in Jα18−/− mice, which lack iNKT cells (28). Whereas an intra-peritoneal injection of a fecal slurry solution (90 mg/mL) in B6 mice resulted in 100% mortality at 24 hours (FIG. 2b), the sham B6 and Jα18−/− groups, which were injected with normal saline, as well as the septic Jα18−/− group, remained alive. On necropsy, we observed discrete abscess collections overlying the intestines and liver in septic Jα18−/− mice, whereas septic B6 mice developed intestinal distension and edema without abscess formation (unpublished observations). In addition, the adoptive transfer of iNKT cells from GFP transgenic mice into Jα18−/− mice and subsequent induction of IAS increased disease severity, in comparison to Jα18−/− mice that did not receive iNKT cells (FIG. 9a). Together, these results confirm the pathogenic nature of iNKT cells in IAS.

Tissue-Specific Distribution of iNKT Cells is Altered in IAS

Previous animal studies using a model of chronic polymicrobial sepsis found that the frequency of hepatic iNKT cells declined significantly, whereas splenic iNKT cells remained unchanged (11). We sought to determine whether a similar occurrence would be observed in IAS. Furthermore, we hypothesized that the omentum, which has been described as the “policeman of the abdomen” for its ability to migrate to and mitigate inflammatory reactions (37) may accommodate increased numbers of iNKT cells post-sepsis.

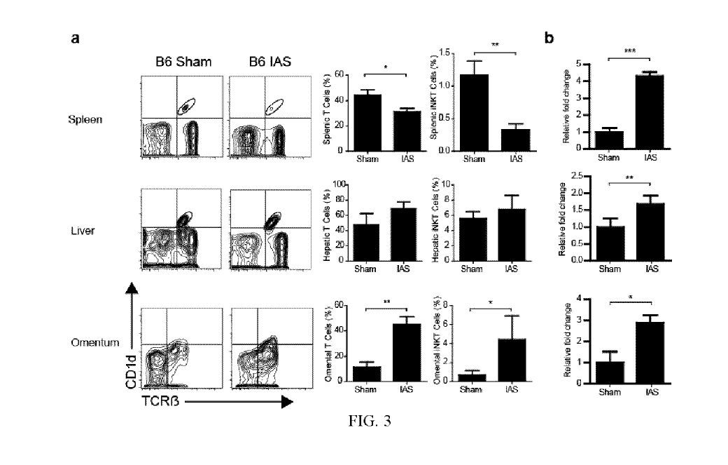

Using flow cytometry, we determined the frequencies of TCRβ+CD1d tetramer—conventional T cells and TCRβ+CD1d tetramer+iNKT cells in the spleen, liver and omentum. In the spleen, the percentages of conventional T cells and iNKT cells declined significantly post-sepsis (FIG. 3a) while there was no difference in the distribution of iNKT or T cells in the liver (FIG. 3a). In the omentum, the percentages of T cells and iNKT cells increased significantly post-sepsis (FIG. 3a). In Jα18−/− mice that were adoptively transferred with iNKT cells from GFP transgenic mice, we detected the presence of these cells within the omentum post-sepsis (FIG. 9b).

We also sought to quantify the transcriptional expression of the invariant TCR following IAS, because the surface receptors of iNKT cells (including TCR and NK1.1) can be down-regulated upon activation (38, 39) and become undetectable by flow cytometry using standard reagents (38). Using the Taqman assay with custom designed primers that overlap the invariant TCR Val 4-Jα18 splice site and amplify a portion of the TCR (40, 41), we observed significant increases in the transcriptional expression of the invariant TCR within the spleen, liver, and omentum post-sepsis (FIG. 3b). Together, these results demonstrate that the tissue-specific distribution of iNKT cells is altered significantly during IAS, and that the transcription of the invariant TCR is increased post-sepsis.

Th2-Polarized iNKT Cells Reduce Disease Severity in IAS

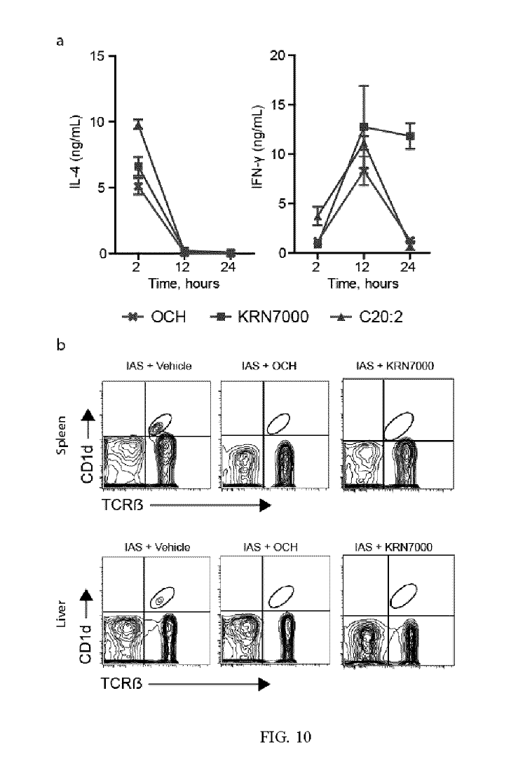

Multiple groups, including ours, have examined the use of glycolipids to modulate cytokine responses in iNKT cells, and ameliorate disease severity in autoimmune diseases such as Type 1 Diabetes (30, 42) and rheumatoid arthritis (6, 8, 43). Since the acute phase of intra-abdominal sepsis is primarily characterized by a marked pro-inflammatory or Th1-type response that contributes to mortality (4, 44-47), we hypothesized that administration of a Th2-polarizing glycolipid would reduce disease severity in sepsis. OCH is an iNKT cell agonist which results in a Th2-biased cytokine profile when administered in vivo (36, 48). Similar to previous studies by our group and others (27, 30, 36), we demonstrated that the intraperitoneal injection of OCH into naïve B6 mice results in a rapid peak of serum IL-4 at 2 hours, and is then significantly reduced at 12 to 24 hours (FIG. 10a); in contrast, serum levels of the Th1 cytokine IFN-γ peaked at 12 hours, but were almost undetectable at 24 hours. The administration of the prototypical iNKT cell agonist KRN7000 (49) resulted in elevated serum levels of IFN-γ between 12 and 24 hours (FIG. 10a). The IL-4: IFN-γ ratio calculated based on the peak values of these cytokines was higher for OCH compared to KRN7000, indicating that OCH promotes a Th2-dominant cytokine response in vivo.

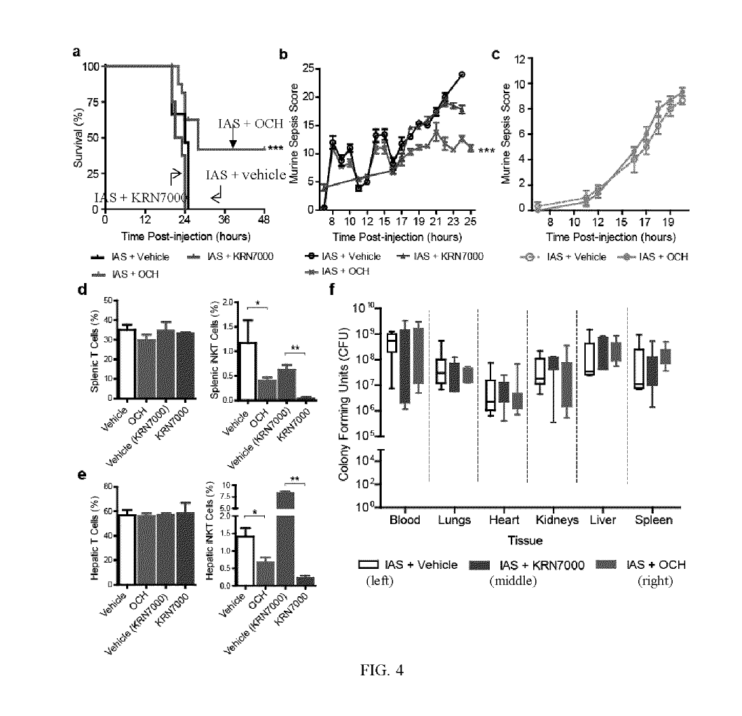

Treatment with OCH prolonged survival in septic mice compared to both vehicle and KRN7000 treatments (FIG. 4a). Median survival for OCH-treated mice was 28 hours compared to 24 and 22 hours for vehicle- and KRN7000-treated mice, respectively (p<0.0001 by log-rank test). Mice in the OCH group survived beyond 24 hours, whereas mortality for vehicle- and KRN7000-treated mice was 100% by 24 hours. OCH-treated mice also had a significantly lower MSS after 24 h compared to vehicle- and KRN7000-treated mice with IAS (FIG. 4b). However, there were no statistical differences in MSS between the vehicle and KRN7000 treatments. The reduced MSS for OCH-treated mice derived from significant improvements in respiratory status, an important clinical predictor of mortality in sepsis (50-53). Most vehicle- and KRN7000-treated mice developed respiratory distress (laboured breathing and reduced respiratory rates) by 15 hours post-sepsis, unlike OCH-treated mice that continued to have relatively normal respiratory rates even at 24 hours. OCH-treated mice were also more responsive to auditory and touch stimuli whereas vehicle- and KRN7000-treated mice remained non-responsive and slow-moving or stationary. In addition, we did not observe any differences in disease severity between vehicle- and OCH-treated Jα18−/− mice with IAS (FIG. 4c), consistent with the mechanism for the beneficial effects of OCH on sepsis severity and mortality in B6 mice being linked to the specific modulation of iNKT cells.

Next, we analyzed the spleens and livers of septic mice treated with the glycolipid agonists but did not detect differences in splenic or hepatic T cell distributions (FIG. 4d and 4e, respectively). However, we had significantly reduced detection of iNKT cells in the spleen and liver following glycolipid treatment (FIG. 4d and 4e). This reflects the down-regulation of the surface TCR that occurs with administration of glycolipid agonists, as shown previously by our group and others (8, 27, 36, 54); FIG. 4e and FIG. 10b). In particular, we observed a significantly lower detection of iNKT cells following KRN7000 treatment compared to treatment with OCH (FIG. 4d and 4e). The differential degree to which the glycolipids down-regulate the surface TCR is a reflection of their differential binding kinetics to iNKT cells. While OCH and KRN7000 down-regulate the surface TCR within 4-12 hours post administration, KRN7000 is approximately 10-fold more potent at down-regulating the TCR after 24 hours (54), leading to the results we observed in FIGS. 4d and 4e.

Anti-inflammatory processes are concomitantly initiated to mitigate pro-inflammatory states in sepsis, both systemically (55-58), and in individual organs (59). These immunosuppressive mechanisms decrease the responsiveness of cells of the innate and adaptive immune systems, thereby increasing susceptibility to opportunistic and additional infections (60-63). Importantly, we observed that the use of OCH, which significantly reduced the production of the pro-inflammatory cytokine IFN-γ (8, 30, 36), did not worsen the microbial load of septic mice, compared to vehicle and KRN7000 treatments (FIG. 4f). Therefore, administration of the Th2-polarizing glycolipid OCH did not result in overt susceptibility to microbial infection. Additionally, OCH-treated mice that survived to 48 hours demonstrated a significantly lower bacterial count in all tested organs, compared to OCH-treated mice that died at 24 hours (data not shown). Sham mice, as expected, did not demonstrate bacterial organ counts (data not shown).

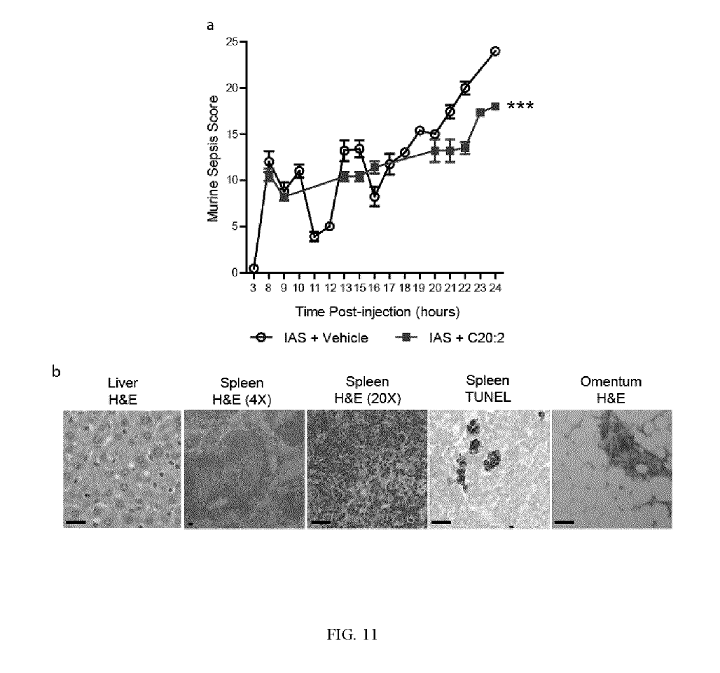

Lastly, we tested the effect of a second Th2-polarizing glycolipid C20:2 on disease severity in IAS, to confirm whether the Th2-biased modulation of iNKT cells was responsible for ameliorating disease severity. C20:2 is a potent agonist with a capacity to bind and activate iNKT cells that is significantly stronger than OCH (30, 36); administration of C20:2 in naïve B6 mice also results in a more pronounced Th2 response at 24 hours than OCH (30, 36) (FIG. 10a). When septic B6 mice were treated with C20:2, we observed a significant reduction in MSS between 20 and 24 hours compared to vehicle-treated mice (FIG. 11a), with improved respiratory status at observed time points. These results confirm the novelty of manipulating iNKT cells into a Th2-biased state for the mitigation of disease severity in IAS. However, the MSS continued to rise in C20:2-treated mice, in contrast to OCH, where the MSS reached a plateau (FIG. 4b). Based on these results, we elected to focus on OCH and the means by which it improves mortality in IAS. However, C20:2 remains a valid treatment option.

The Pro-Inflammatory Cytokine Profile in IAS is Ameliorated by Administration of OCH

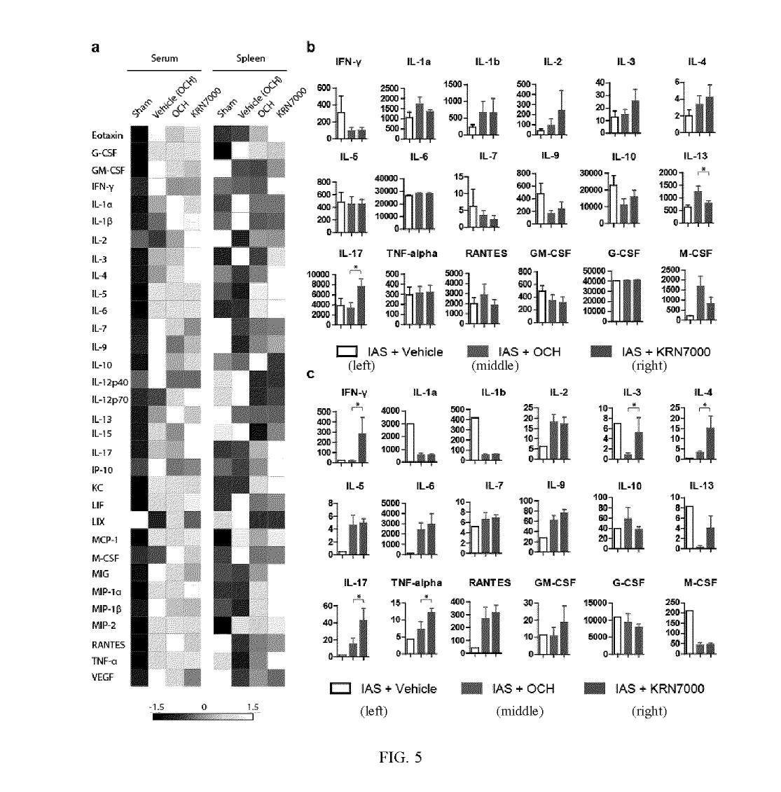

In order to further understand the impact of the glycolipid agonists on the septic response, we assessed the concentrations of 32 cytokines and chemokines from the sera and spleens of vehicle-, OCH-, and KRN7000-treated septic mice, as well as sham treated mice (FIG. 5a-c). In the serum, mean concentrations of IL-17 were significantly lower in the OCH-treated mice compared to KRN7000-treated mice. The concentration of IL-13 was higher in the sera of OCH-treated mice compared to KRN7000-treated and vehicle-treated mice. In the spleen, IFN-γ, IL-3, IL-4, IL-17, and TNF-α were significantly elevated in the KRN7000-treated group compared to the OCH-treated group. Therefore, the administration of OCH significantly reduces the levels of pro-inflammatory cytokines in IAS.

Treatment with OCH Significantly Reduces Splenocyte Apoptosis in IAS

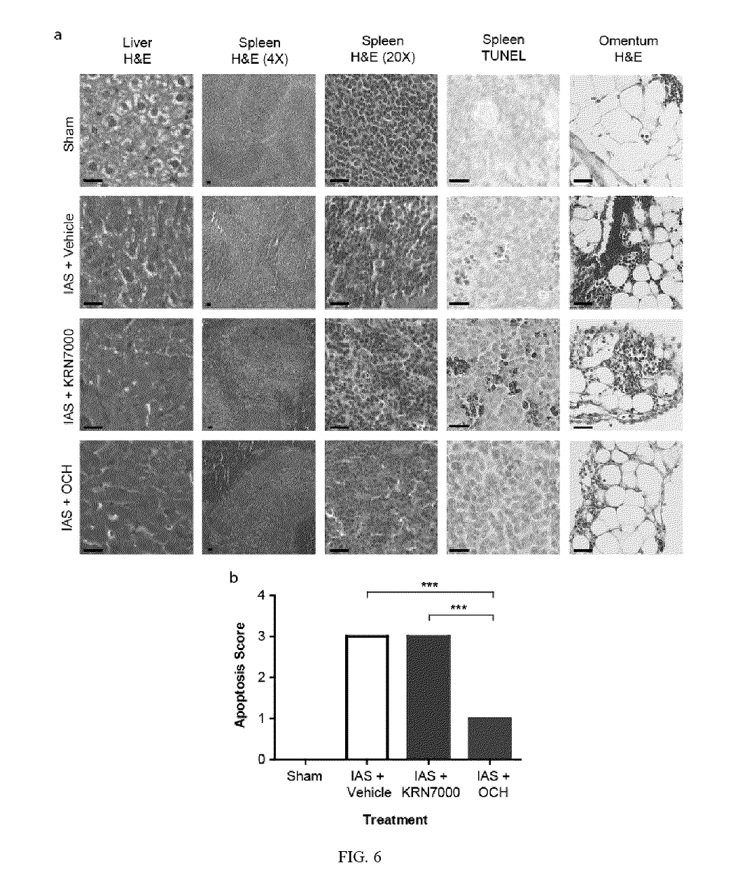

We next sought to elucidate the reason for the improved survival among septic mice that were treated with OCH. When we performed histopathological analysis on the spleen, liver, and omentum of septic B6 mice treated with KRN7000 or OCH (FIG. 6a), we found a significant reduction of apoptotic cells within the spleens of OCH-treated mice compared to vehicle- and KRN7000-treated mice. The presence of karyorrhexic nuclei within clusters of cells with eosinophilic cytoplasm was observed in the white pulp of the spleen by hematoxylin and eosin staining, and subsequently confirmed as apoptotic cells by TUNEL staining, particularly in vehicle- and KRN7000-treated mice. Based on histopathological scoring, OCH-treated mice had mild apoptosis, whereas vehicle-treated and KRN7000-treated mice had moderate and severe apoptosis respectively (FIG. 6b).

In the omentum of vehicle- and KRN7000-treated mice, we noted a significant increase in lymphocytes whereas fewer lymphocytes were observed in the omentum of OCH-treated mice (FIG. 6a). We did not observe overt differences in liver histopathology among vehicle-, OCH-, and KRN-treated mice. When we examined the histology of C20:2-treated septic mice, we observed a decrease in apoptosis compared to KRN7000-treated mice. However, the degree of apoptosis in C20:2-treated mice were higher than OCH-treated mice with IAS (FIG. 11b).

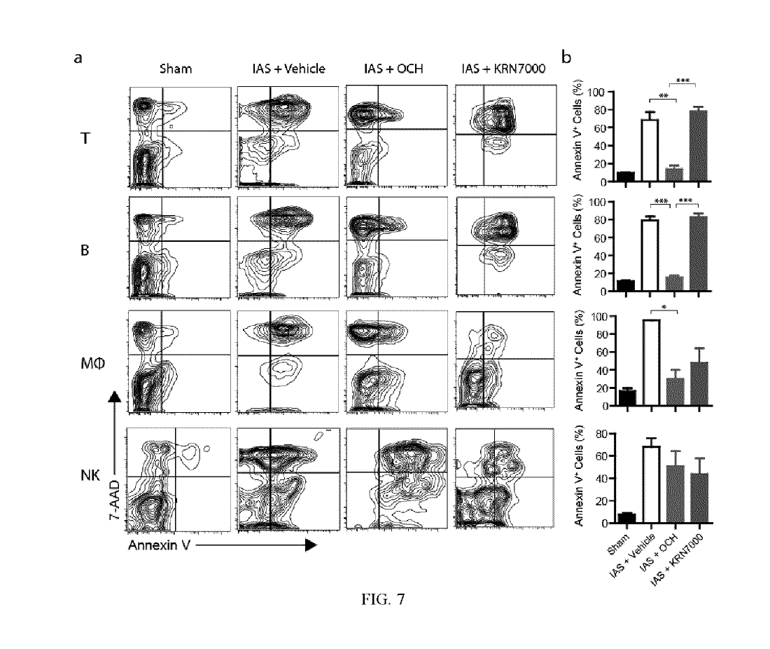

We then performed flow cytometry on spleens harvested from vehicle-, OCH- and KRN7000-treated mice with IAS to determine the immune cell populations that had undergone apoptosis (FIG. 7). Treatment with OCH significantly reduced the apoptosis of T and B cells compared to vehicle- and KRN7000-treated mice. However, there were no differences in the frequency of apoptotic macrophages between the KRN7000 and OCH groups, although both treatments reduced the frequency of apoptosis significantly compared to vehicle-treated mice. With respect to NK cell apoptosis, we observed a trend toward reduced apoptosis in KRN7000-treated mice. Together, these results demonstrate that different glycolipid agonists of iNKT cells differentially mitigate the apoptosis of splenic lymphocytes, but not NK cells and macrophages. Moreover, Th2-polarizing glycolipids significantly reduce lymphocyte apoptosis within the spleen, a critical predictor of mortality in severe sepsis and septic shock (64-66).

Discussion

iNKT cells exert profound and diverse regulatory functions in health and disease, bridging the innate and adaptive defense mechanisms in a variety of immune responses (10, 14, 67). Here, we demonstrate that patients with sepsis/severe sepsis have significantly elevated proportions of iNKT cells and that OCH, a Th2-polarizing glycolipid agonist of iNKT cells, substantially reduces disease severity in acute IAS, with significantly reduced lymphocyte apoptosis within the spleen. These findings introduce iNKT cells as therapeutic targets for the treatment of sepsis in subjects.

Glycolipid ligands of iNKT cells have been used successfully in experimental models of autoimmune diseases (8, 30, 42, 43), transplantation (19, 21), and malignancy (28, 68). KRN7000 (49) reduced morbidity and mortality associated with murine graft-versus-host disease (21, 69), while OCH mitigated disease severity in non-obese diabetic mice (42), experimental autoimmune encephalomyelitis (48), and collagen-induced arthritis (6, 43). OCH also prevented disease symptoms in a humanized mouse model of citrullinated fibrinogen-induced inflammatory arthritis (8), and delayed Th1-mediated cardiac allograft rejection in mice (19).

In the present study, we show that the administration of OCH ameliorated the severe pro-inflammatory Th1-type response associated with IAS and reduced mortality. Although pro-inflammatory cytokines such as IFN-γ and TNF-α contribute to immune responses against bacterial infections (70), elevated levels of these cytokines are also associated with poor outcomes and decreased survival in sepsis (71, 72; Shrum et al. BMC Research Notes 2014, 7:233). As confirmed in this study, the treatment of septic mice with KRN7000 resulted in a Th1-type response at 24 hours (10, 36, 73) and did not affect disease severity. In addition, elevated levels of the Th2 cytokine, IL-13, may be contributing to the significant improvements in respiratory status and disease severity that we observed in OCH-treated mice. A potent anti-inflammatory cytokine (74, 75), IL-13 is produced in large quantities by alveolar macrophages in the lung during polymicrobial sepsis (74), and has been shown to protect mice from endotoxic shock when administered in vivo (76). Since a compromised respiratory status significantly increases morbidity and mortality in sepsis (50-52), the selective Th2-biased modulation of iNKT cells may provide a novel strategy to prevent this complication.

The relative deficiency of pro-inflammatory cytokines has also been associated with increased susceptibility to additional infections. However, we did not observe an increase in microbial load within the blood and organs of OCH-treated mice compared to vehicle-treated mice with IAS. Since OCH is a less potent agonist than KRN7000, with lower binding affinity for the invariant TCR compared to the latter (36, 54), the administration of a single dose of OCH may have affected only a portion of iNKT cells, thereby abrogating rather than eliminating the pro-inflammatory response. In addition, other immune cells which are not directly affected by glycolipid administration may continue to participate in bacterial clearance, including NK cells, which also produce significant amounts of IFN-γ (70). Any differences in microbial counts between KRN7000- and vehicle-treated mice may have been masked by the excessive pro-inflammatory response that is inherent in our sepsis model (Shrum et al, submitted for publication). Lastly, our study also confirms that the manipulation of iNKT cells alone can dramatically alter outcomes in sepsis, given that iNKT-deficient mice are resistant to mortality from sepsis, and disease severity was unaffected by glycolipid treatment in these animals.

Interestingly, the use of C20:2, another Th2-polarizing glycolipid that is significantly more potent at inducing a Th2 bias compared to OCH (30, 31, 77) and suppresses downstream NK cell function (36), also mitigated sepsis severity significantly. Unlike OCH, however, C20:2-treated mice continued to worsen, while the histopathology of the spleen demonstrated a higher degree of apoptosis following treatment with C20:2 compared to OCH; these findings may be explained by the relatively short half-life of C20:2 compared to OCH (30, 36). Nevertheless, the results of our study suggest that the Th2-biased manipulation of iNKT cells may be a viable therapeutic strategy in sepsis, although optimization of the timing and frequency of glycolipid usage may be needed to provide the most effective results.

We also show that the tissue-specific distribution of iNKT cells is altered during IAS, with significant reductions in the spleen, and a concomitant rise in the omentum. The human omentum has been described as the “policeman of the abdomen” for its ability to adhere to sites of intra-abdominal pathology and prevent widespread pathogen contamination (78, 79). Similarly, the murine omentum has been shown to facilitate the regeneration of damaged tissues (37). These results, as well as the findings of Lynch et al., who demonstrated that the human omentum contained a rich reservoir of iNKT cells (80), prompted us to examine the murine omentum, wherein we observed a significant increase in iNKT cells post-sepsis. Our observation that the omentum became enlarged during IAS correlates with findings by Shah et al. (37), and represents a unique feature of this organ that has not been noted in other secondary lymphoid structures such as lymph nodes or spleens. T cells were also noted to be significantly increased in the omentum during IAS, corroborating observations made by Carlow et al. (81) in a cecal ligation and puncture (CLP) model of polymicrobial sepsis. Our results with respect to the tissue-specific distribution of iNKT cells post-sepsis contrast with the findings of Hu et al. (11), who demonstrated a significant reduction in hepatic iNKT cells but no changes in the frequency of splenic iNKT cells in the CLP model. We propose that splenic iNKT cells mobilize more readily during acute sepsis compared to hepatic iNKT cells, since a recent study by Barral et al. (13) showed that splenic iNKT cells patrol the red pulp and marginal zones of the spleen, rapidly sample blood-borne antigens, and display migratory capabilities. This may explain our observed changes in splenic iNKT cell frequency post-sepsis, and additionally suggests that the iNKT cells we detected in the omentum post-sepsis may have originated from the spleen, given that the two organs are physically attached to each other (37).

In this study, we also demonstrate that Th2-polarized iNKT cells significantly reduce apoptosis within the spleen, particularly among T and B cells as well as macrophages. iNKT cells can rapidly sense, and are activated by, apoptotic cell death (82, 83). Wermeling et al. (83) showed that in B6 mice injected with apoptotic cells, activated iNKT cells reduce B cell survival. Moreover, the cytokine profile of these iNKT cells was altered towards a Th2-type response, albeit in ex vivo splenocyte cultures rather than in vivo (83). Our group has previously demonstrated that Th2-polarized iNKT cells undergo less apoptosis in a model of autoimmune diabetes compared to Th1-biased iNKT cells (31). No studies to date, however, have examined the impact of modulating iNKT cell phenotype on sepsis-induced apoptosis. The latter is an especially important phenomenon with significant immunological and clinical implications. The apoptosis of T and B cells significantly impairs the adaptive immune response, and, by disabling cross-talk between the adaptive and innate immune systems, also impairs the latter (5, 59, 84). These mechanisms lead to an immunosuppressive phase in septic patients, which may result to additional secondary infections that substantially increase mortality (5, 84). Hotchkiss et al. observed a striking apoptosis-induced loss of cells of the innate and adaptive immune systems in the spleen during sepsis, including CD4+ and CD8+ T cells, B cells, and dendritic cells (44, 66). Additionally, T cells that come into contact with macrophages and dendritic cells that have ingested apoptotic cells either become anergic or undergo apoptosis themselves (85). Therefore, the significant reduction in splenic lymphocyte apoptosis following treatment with OCH may preserve the function and efficacy of immune cells, prevent anergy, and mitigate the immunosuppressive phase during sepsis. Interestingly, apoptosis of NK cells appeared to be reduced by treatment with OCH and KRN7000, although the trend is more pronounced for the latter. Since NK cells also produce significant amounts of IFN-γ(70), their apoptosis in the spleens of vehicle- and OCH-treated mice may explain the reduced levels of splenic IFN-γ in these two groups.

We have also demonstrated that the proportion of circulating iNKT cells is elevated early in the septic process for critically-ill patients, corroborating a recently published study by Heffernan et al. (86). Given their propensity to rapidly produce significant quantities of pro- and/or anti-inflammatory cytokines, the increased proportion of iNKT cells suggests that these cells may play a prominent role in promulgating the immune response in septic patients. Furthermore, we observed that the proportion of iNKT cells is not increased in patients who have sustained significant inflammatory responses due to trauma, suggesting that these cells may be specifically responding to microbial pathogens in humans. Consequently, the detection of increased numbers of iNKT cells may also serve as an important biomarker to differentiate septic from non-septic patients early in the disease process, thereby facilitating rapid and targeted interventions for the disease.

Given the failure of many immunotherapeutic drugs in the treatment of sepsis (87, 88), alternative agents have been sought to combat this disease with some success (89-95). The results of our study demonstrate that iNKT cells should be further considered as potential targets for immunotherapy in sepsis, and that modulation of iNKT cell phenotype towards a Th2 response has a protective effect during acute infection.

TABLE 1
Demographics and clinical characteristics of patients with
and without sepsis

| | Non-septic | Septic | |
|---|---|---|---|
| Demographic and clinical characteristics | (n = 7) | (n = 23) | P Value |
| Median Age (y) | 61 | 59 | 0.433 |
| Gender, n (%): | | | <0.0001 |
| Male | 6 (85) | 13 (57) | |
| Female | 1 (15) | 10 (43) | |
| Mean APACHEII Score, n | 23 | 16 | 0.377 |
| Disease severity on admission, n (%): | | | |
| Sepsis | — | 16 (69) | — |
| Severe Sepsis | — | 7 (31) | — |
| Septic Shock | — | 0 (0) | — |
| Source of infection, n (%): | | | |
| Intra-abdominal | — | 10 (44) | — |
| Pneumonia | — | 9 (39) | — |
| Other¹ | — | 4 (17) | — |
| Documented microbial agent, n (%): | | | |
| Gram-positive | — | 7 (30) | — |
| Gram-negative | — | 4 (17) | — |
| Fungi | — | 1 (4) | — |
| Polymicrobial | — | 7 (30) | — |
| None/Unknown | — | 4 (17) | — |

¹Includes patients with skin and soft-tissue infections (2) and urosepsis (2)

TABLE 2
Comparison of outcomes among patients with and without sepsis.

| Variable | Non-Septic | Septic | P Value |
|---|---|---|---|
| Median white blood cell count | 10.6 | 11.5 | 0.182 |
| (×10⁹/L) | | | |
| Lymphocytes¹, % | 16.2 | 17.6 | 0.252 |
| Lymphocyte subsets², %: | | | |
| T cells | 36.7 | 57.8 | 0.039 |
| NK cells | 5.19 | 12.25 | 0.274 |
| iNKT cells | 0.0041 | 0.0057 | 0.138 |
| iNKT:T cell ratio | 0.009 | 0.020 | 0.047 |
| Mean hospital stay (range), d | 12.8 (0-38) | 25.2 (4-55) | 0.045 |
| In-hospital mortality, n (%) | 3 (43) | 5 (28) | 0.955 |
| Cause of death, n (%): | | | 0.293 |
| Multi-organ failure | 1 (14) | 4 (17) | |
| Cardiac arrest | 1 (14) | 0 (0) | |
| Withdrawal of care | 1 (14) | 1 (4) | |

¹Expressed as a percentage of the total sample analyzed on flow cytometry
²Expressed as a percentage of lymphocytes. Median populations are presented.

### REFERENCES

1. Bone, R. C., R. A. Balk, F. B. Cerra, R. P. Dellinger, A. M. Fein, W. A. Knaus, R. M. Schein, and W. J. Sibbald. 2009. Definitions for sepsis and organ failure and guidelines for the use of innovative therapies in sepsis. The ACCP/SCCM Consensus Conference Committee. American College of Chest Physicians/Society of Critical Care Medicine. 1992. Chest 136: e28.
2. Angus, D. C., W. T. Linde-Zwirble, J. Lidicker, G. Clermont, J. Carcillo, and M. R. Pinsky. 2001. Epidemiology of severe sepsis in the United States: analysis of incidence, outcome, and associated costs of care. Critical care medicine 29: 1303-1310.
3. Parrillo, J. E., M. M. Parker, C. Natanson, A. F. Suffredini, R. L. Danner, R. E. Cunnion, and F. P. Ognibene. 1990. Septic shock in humans. Advances in the understanding of pathogenesis, cardiovascular dysfunction, and therapy. Annals of internal medicine 113: 227-242.
4. Hotchkiss, R. S., and I. E. Karl. 2003. The pathophysiology and treatment of sepsis. The New England journal of medicine 348: 138-150.
5. Hotchkiss, R. S., G. Monneret, and D. Payen. 2013. Immunosuppression in sepsis: a novel understanding of the disorder and a new therapeutic approach. The Lancet Infectious Diseases 13: 260-268.
6. Miellot-Gafsou, A., J. Biton, E. Bourgeois, A. Herbelin, M. C. Boissier, and N. Bessis. 2010. Early activation of invariant natural killer T cells in a rheumatoid arthritis model and application to disease treatment. Immunology 130: 296-306.
7. Van Kaer, L. 2005. alpha-Galactosylceramide therapy for autoimmune diseases: prospects and obstacles. Nature reviews. Immunology 5: 31-42.
8. Walker, K. M., M. Rytelewski, D. M. Mazzuca, S. A. Meilleur, L. A. Mannik, D. Yue, W. C. Brintnell, I. Welch, E. Cairns, and S. M. Haeryfar. 2012. Preventing and curing citrulline-induced autoimmune arthritis in a humanized mouse model using a Th2-polarizing iNKT cell agonist. Immunology and cell biology 90: 630-639.
9. Bendelac, A., P. B. Savage, and L. Teyton. 2007. The biology of NKT cells. Annual review of immunology 25: 297-336.
10. Godfrey, D. I., H. R. MacDonald, M. Kronenberg, M. J. Smyth, and L. Van Kaer. 2004. NKT cells: what's in a name? Nature reviews. Immunology 4: 231-237.
11. Hu, C. K., F. Venet, D. S. Heffernan, Y. L. Wang, B. Horner, X. Huang, C. S. Chung, S. H. Gregory, and A. Ayala. 2009. The role of hepatic invariant NKT cells in systemic/local inflammation and mortality during polymicrobial septic shock. Journal of immunology (Baltimore, Md.: 1950) 182: 2467-2475.
12. Kinjo, Y., P. Illarionov, J. L. Vela, B. Pei, E. Girardi, X. Li, Y. Li, M. Imamura, Y. Kaneko, A. Okawara, Y. Miyazaki, A. Gomez-Velasco, P. Rogers, S. Dahesh, S. Uchiyama, A. Khurana, K. Kawahara, H. Yesilkaya, P. W. Andrew, C. H. Wong, K. Kawakami, V. Nizet, G. S. Besra, M. Tsuji, D. M. Zajonc, and M. Kronenberg. 2011. Invariant natural killer T cells recognize glycolipids from pathogenic Gram-positive bacteria. Nature immunology 12: 966-974.
13. Barral, P., M. D. Sanchez-Nino, N. van Rooijen, V. Cerundolo, and F. D. Batista. 2012. The location of splenic NKT cells favours their rapid activation by blood-borne antigen. The EMBO journal 31: 2378-2390.
14. Kronenberg, M. 2005. Toward an understanding of NKT cell biology: progress and paradoxes. Annual review of immunology 23: 877-900.
15. Dieli, F., G. Sireci, D. Russo, M. Taniguchi, J. Ivanyi, C. Fernandez, M. Troye-Blomberg, G. De Leo, and A. Salerno. 2000. Resistance of natural killer T cell-deficient mice to systemic Shwartzman reaction. The Journal of experimental medicine 192: 1645-1652.
16. Brigl, M., L. Bry, S. C. Kent, J. E. Gumperz, and M. B. Brenner. 2003. Mechanism of CD1d-restricted natural killer T cell activation during microbial infection. Nature immunology 4: 1230-1237.
17. Skold, M., and S. M. Behar. 2003. Role of CD1d-restricted NKT cells in microbial immunity. Infection and immunity 71: 5447-5455.
18. Leung, B., and H. W. Harris. 2011. NKT cells: the culprits of sepsis? The Journal of surgical research 167: 87-95.
19. Haeryfar, S. M., Z. Lan, M. Leon-Ponte, K. R. Duffy, W. Ge, W. Liu, T. Mele, B. Garcia, and H. Wang. 2008. Prolongation of cardiac allograft survival by rapamycin and the invariant natural killer T cell glycolipid agonist OCH. Transplantation 86: 460-468.
20. Parekh, V. V., M. T. Wilson, D. Olivares-Villagomez, A. K. Singh, L. Wu, C. R. Wang, S. Joyce, and L. Van Kaer. 2005. Glycolipid antigen induces long-term natural killer T cell anergy in mice. The Journal of clinical investigation 115: 2572-2583.
21. Hashimoto, D., S. Asakura, S. Miyake, T. Yamamura, L. Van Kaer, C. Liu, M. Tanimoto, and T. Teshima. 2005. Stimulation of host NKT cells by synthetic glycolipid regulates acute graft-versus-host disease by inducing Th2 polarization of donor T cells. Journal of immunology (Baltimore, Md.: 1950) 174: 551-556.
22. Oki, S., A. Chiba, T. Yamamura, and S. Miyake. 2004. The clinical implication and molecular mechanism of preferential IL-4 production by modified glycolipid-stimulated NKT cells. The Journal of clinical investigation 113: 1631-1640.
23. World Medical, A. 2013. World Medical Association Declaration of Helsinki: ethical principles for medical research involving human subjects. JAMA: the journal of the American Medical Association 310: 2191-2194.
24. Levy, M. M., M. P. Fink, J. C. Marshall, E. Abraham, D. Angus, D. Cook, J. Cohen, S. M. Opal, J. L. Vincent, and G. Ramsay. 2003. 2001 SCCM/ESICM/ACCP/ATS/SIS International Sepsis Definitions Conference. Critical care medicine 31: 1250-1256.
25. Knaus, W. A., E. A. Draper, D. P. Wagner, and J. E. Zimmerman. 1985. APACHE II: a severity of disease classification system. Critical care medicine 13: 818-829.
26. Knaus, W. A., J. E. Zimmerman, D. P. Wagner, E. A. Draper, and D. E. Lawrence. 1981. APACHE-acute physiology and chronic health evaluation: a physiologically based classification system. Critical care medicine 9: 591-597.
27. Hayworth, J. L., D. M. Mazzuca, S. Maleki Vareki, I. Welch, J. K. McCormick, and S. M. Haeryfar. 2012. CD1d-independent activation of mouse and human iNKT cells by bacterial superantigens. Immunology and cell biology 90: 699-709.
28. Cui, J., T. Shin, T. Kawano, H. Sato, E. Kondo, I. Toura, Y. Kaneko, H. Koseki, M. Kanno, and M. Taniguchi. 1997. Requirement for Valpha14 NKT cells in IL-12-mediated rejection of tumors. Science (New York, N.Y.) 278: 1623-1626.
29. Goff, R. D., Y. Gao, J. Mattner, D. Zhou, N. Yin, C. Cantu, 3rd, L. Teyton, A. Bendelac, and P. B. Savage. 2004. Effects of lipid chain lengths in alpha-galactosylceramides on cytokine release by natural killer T cells. Journal of the American Chemical Society 126: 13602-13603.
30. Ly, D., R. Tohn, B. Rubin, H. Blumenfeld, G. S. Besra, N. Veerapen, S. A. Porcelli, and T. L. Delovitch. 2010. An alpha-galactosylceramide C20:2 N-acyl variant enhances anti-inflammatory and regulatory T cell-independent responses that prevent type 1 diabetes. Clinical and experimental immunology 160: 185-198.
31. Tohn, R., H. Blumenfeld, S. M. Haeryfar, N. Veerapen, G. S. Besra, S. A. Porcelli, and T. L. Delovitch. 2011. Stimulation of a shorter duration in the state of anergy by an invariant natural killer T cell agonist enhances its efficiency of protection from type 1 diabetes. Clinical and experimental immunology 164: 26-41.
32. Bustin, S. A., V. Benes, J. A. Garson, J. Hellemans, J. Huggett, M. Kubista, R. Mueller, T. Nolan, M. W. Pfaffl, G. L. Shipley, J. Vandesompele, and C. T. Wittwer. 2009. The MIQE guidelines: minimum information for publication of quantitative real-time PCR experiments. Clinical chemistry 55: 611-622.
33. Pavlidis, P., and W. S. Noble. 2003. Matrix2png: a utility for visualizing matrix data. Bioinformatics (Oxford, England) 19: 295-296.
34. Elmore, S. 2007. Apoptosis: a review of programmed cell death. Toxicologic pathology 35: 495-516.
35. Stetson, D. B., M. Mohrs, R. L. Reinhardt, J. L. Baron, Z. E. Wang, L. Gapin, M. Kronenberg, and R. M. Locksley. 2003. Constitutive cytokine mRNAs mark natural killer (NK) and NK T cells poised for rapid effector function. The Journal of experimental medicine 198: 1069-1076.
36. Yu, K. O., J. S. Im, A. Molano, Y. Dutronc, P. A. Illarionov, C. Forestier, N. Fujiwara, I. Arias, S. Miyake, T. Yamamura, Y. T. Chang, G. S. Besra, and S. A. Porcelli. 2005. Modulation of CD1d-restricted NKT cell responses by using N-acyl variants of alpha-galactosylceramides. Proceedings of the National Academy of Sciences of the United States of America 102: 3383-3388.
37. Shah, S., E. Lowery, R. K. Braun, A. Martin, N. Huang, M. Medina, P. Sethupathi, Y. Seki, M. Takami, K. Byrne, C. Wigfield, R. B. Love, and M. Iwashima. 2012. Cellular basis of tissue regeneration by omentum. PloS one 7: e38368.
38. Wilson, M. T., C. Johansson, D. Olivares-Villagomez, A. K. Singh, A. K. Stanic, C. R. Wang, S. Joyce, M. J. Wick, and L. Van Kaer. 2003. The response of natural killer T cells to glycolipid antigens is characterized by surface receptor down-modulation and expansion. Proceedings of the National Academy of Sciences of the United States of America 100: 10913-10918.
39. Harada, M., K. Seino, H. Wakao, S. Sakata, Y. Ishizuka, T. Ito, S. Kojo, T. Nakayama, and M. Taniguchi. 2004. Down-regulation of the invariant Valpha14 antigen receptor in NKT cells upon activation. International immunology 16: 241-247.
40. Matsuda, J. L., L. Gapin, J. L. Baron, S. Sidobre, D. B. Stetson, M. Mohrs, R. M. Locksley, and M. Kronenberg. 2003. Mouse V alpha 14i natural killer T cells are resistant to cytokine polarization in vivo. Proceedings of the National Academy of Sciences of the United States of America 100: 8395-8400.
41. Gapin, L., J. L. Matsuda, C. D. Surh, and M. Kronenberg. 2001. NKT cells derive from double-positive thymocytes that are positively selected by CD1d. Nature immunology 2: 971-978.
42. Mizuno, M., M. Masumura, C. Tomi, A. Chiba, S. Oki, T. Yamamura, and S. Miyake. 2004. Synthetic glycolipid OCH prevents insulitis and diabetes in NOD mice. Journal of autoimmunity 23: 293-300.
43. Chiba, A., S. Oki, K. Miyamoto, H. Hashimoto, T. Yamamura, and S. Miyake. 2004. Suppression of collagen-induced arthritis by natural killer T cell activation with OCH, a sphingosine-truncated analog of alpha-galactosylceramide. Arthritis and rheumatism 50: 305-313.
44. Hotchkiss, R. S., P. E. Swanson, B. D. Freeman, K. W. Tinsley, J. P. Cobb, G. M. Matuschak, T. G. Buchman, and I. E. Karl. 1999. Apoptotic cell death in patients with sepsis, shock, and multiple organ dysfunction. Critical care medicine 27: 1230-1251.
45. Alberti, C., C. Brun-Buisson, S. Chevret, M. Antonelli, S. V. Goodman, C. Martin, R. Moreno, A. R. Ochagavia, M. Palazzo, K. Werdan, and J. R. Le Gall. 2005. Systemic inflammatory response and progression to severe sepsis in critically ill infected patients. American journal of respiratory and critical care medicine 171: 461-468.
46. Riedemann, N. C., R. F. Guo, and P. A. Ward. 2003. Novel strategies for the treatment of sepsis. Nature medicine 9: 517-524.
47. Ulloa, L., and K. J. Tracey. 2005. The “cytokine profile”: a code for sepsis. Trends in molecular medicine 11: 56-63.
48. Miyamoto, K., S. Miyake, and T. Yamamura. 2001. A synthetic glycolipid prevents autoimmune encephalomyelitis by inducing TH2 bias of natural killer T cells. Nature 413: 531-534.
49. Kawano, T., J. Cui, Y. Koezuka, I. Toura, Y. Kaneko, K. Motoki, H. Ueno, R. Nakagawa, H. Sato, E. Kondo, H. Koseki, and M. Taniguchi. 1997. CD1d-restricted and TCR-mediated activation of valpha14 NKT cells by glycosylceramides. Science (New York, N.Y.) 278: 1626-1629.
50. Esperatti, M., M. Ferrer, V. Giunta, O. T. Ranzani, L. M. Saucedo, G. Li Bassi, F. Blasi, J. Rello, M. S. Niederman, and A. Torres. 2013. Validation of predictors of adverse outcomes in hospital-acquired pneumonia in the ICU. Critical care medicine 41: 2151-2161.
51. Amato, M. B., C. S. Barbas, D. M. Medeiros, R. B. Magaldi, G. P. Schettino, G. Lorenzi-Filho, R. A. Kairalla, D. Deheinzelin, C. Munoz, R. Oliveira, T. Y. Takagaki, and C. R. Carvalho. 1998. Effect of a protective-ventilation strategy on mortality in the acute respiratory distress syndrome. The New England journal of medicine 338: 347-354.
52. Dellinger, R. P., J. M. Carlet, H. Masur, H. Gerlach, T. Calandra, J. Cohen, J. Gea-Banacloche, D. Keh, J. C. Marshall, M. M. Parker, G. Ramsay, J. L. Zimmerman, J. L. Vincent, and M. M. Levy. 2004. Surviving Sepsis Campaign guidelines for management of severe sepsis and septic shock. Critical care medicine 32: 858-873.
53. Wheeler, A. P., and G. R. Bernard. 1999. Treating patients with severe sepsis. The New England journal of medicine 340: 207-214.
54. Stanic, A. K., R. Shashidharamurthy, J. S. Bezbradica, N. Matsuki, Y. Yoshimura, S. Miyake, E. Y. Choi, T. D. Schell, L. Van Kaer, S. S. Tevethia, D. C. Roopenian, T. Yamamura, and S. Joyce. 2003. Another view of T cell antigen recognition: cooperative engagement of glycolipid antigens by Va14Ja18 natural T(iNKT) cell receptor [corrected]. Journal of immunology (Baltimore, Md.: 1950) 171: 4539-4551.
55. Van Dissel, J. T., P. Van Langevelde, R. G. J. Westendorp, K. Kwappenberg, and M. Frölich. 1998. Anti-inflammatory cytokine profile and mortality in febrile patients. Lancet 351: 950-953.
56. Ertel, W., J. P. Kremer, J. Kenney, U. Steckholzer, D. Jarrar, O. Trentz, and F. W. Schildberg. 1995. Downregulation of proinflammatory cytokine release in whole blood from septic patients. Blood 85: 1341-1347.
57. Munoz, C., J. Carlet, C. Fitting, B. Misset, J. P. Bleriot, and J. M. Cavaillon. 1991. Dysregulation of in vitro cytokine production by monocytes during sepsis. Journal of Clinical Investigation 88: 1747-1754.
58. Rigato, O., and R. Salomao. 2003. Impaired production of interferon-gamma and tumor necrosis factor-alpha but not of interleukin 10 in whole blood of patients with sepsis. Shock (Augusta, Ga.) 19: 113-116.
59. Boomer, J. S., K. To, K. C. Chang, O. Takasu, D. F. Osborne, A. H. Walton, T. L. Bricker, S. D. Jarman Ii, D. Kreisel, A. S. Krupnick, A. Srivastava, P. E. Swanson, J. M. Green, and R. S. Hotchkiss. 2011. Immunosuppression in patients who die of sepsis and multiple organ failure. JAMA—Journal of the American Medical Association 306: 2594-2605.
60. Otto, G., M. Sossdorf, R. Claus, J. Rodel, K. Menge, K. Reinhart, M. Bauer, and N. Riedemann. 2011. The late phase of sepsis is characterized by an increased microbiological burden and death rate. Critical Care 15: R183.
61. Kollef, K. E., G. E. Schramm, A. R. Wills, R. M. Reichley, S. T. Micek, and M. H. Kollef. 2008. Predictors of 30-day mortality and hospital costs in patients with ventilator-associated pneumonia attributed to potentially antibiotic-resistant gram-negative bacteria. Chest 134: 281-287.
62. Limaye, A. P., K. A. Kirby, G. D. Rubenfeld, W. M. Leisenring, E. M. Bulger, M. J. Neff, N. S. Gibran, M. L. Huang, T. K. Santo Hayes, L. Corey, and M. Boeckh. 2008. Cytomegalovirus reactivation in critically ill immunocompetent patients. JAMA: the journal of the American Medical Association 300: 413-422.
63. Luyt, C. E., A. Combes, C. Deback, M. H. Aubriot-Lorton, A. Nieszkowska, J. L. Trouillet, F. Capron, H. Agut, C. Gibert, and J. Chastre. 2007. Herpes simplex virus lung infection in patients undergoing prolonged mechanical ventilation. American journal of respiratory and critical care medicine 175: 935-942.
64. Hotchkiss, R. S., and D. W. Nicholson. 2006. Apoptosis and caspases regulate death and inflammation in sepsis. Nature reviews. Immunology 6: 813-822.
65. Hotchkiss, R. S., K. W. Tinsley, P. E. Swanson, K. C. Chang, J. P. Cobb, T. G. Buchman, S. J. Korsmeyer, and I. E. Karl. 1999. Prevention of lymphocyte cell death in sepsis improves survival in mice. Proceedings of the National Academy of Sciences of the United States of America 96: 14541-14546.
66. Hotchkiss, R. S., K. W. Tinsley, P. E. Swanson, R. E. Schmieg, Jr., J. J. Hui, K. C. Chang, D. F. Osborne, B. D. Freeman, J. P. Cobb, T. G. Buchman, and I. E. Karl. 2001. Sepsis-induced apoptosis causes progressive profound depletion of B and CD4+ T lymphocytes in humans. Journal of immunology (Baltimore, Md.: 1950) 166: 6952-6963.
67. Taniguchi, M., K. Seino, and T. Nakayama. 2003. The NKT cell system: bridging innate and acquired immunity. Nature immunology 4: 1164-1165.
68. Smyth, M. J., K. Y. Thia, S. E. Street, E. Cretney, J. A. Trapani, M. Taniguchi, T. Kawano, S. B. Pelikan, N. Y. Crowe, and D. I. Godfrey. 2000. Differential tumor surveillance by natural killer (NK) and NKT cells. The Journal of experimental medicine 191: 661-668.
69. Morecki, S., S. Panigrahi, G. Pizov, E. Yacovlev, Y. Gelfand, O. Eizik, and S. Slavin. 2004. Effect of KRN7000 on induced graft-vs-host disease. Experimental hematology 32: 630-637.
70. Nakano, Y., K. Onozuka, Y. Terada, H. Shinomiya, and M. Nakano. 1990. Protective effect of recombinant tumor necrosis factor-alpha in murine salmonellosis. Journal of immunology (Baltimore, Md.: 1950) 144: 1935-1941.
71. Doherty, G. M., J. R. Lange, H. N. Langstein, H. R. Alexander, C. M. Buresh, and J. A. Norton. 1992. Evidence for IFN-gamma as a mediator of the lethality of endotoxin and tumor necrosis factor-alpha. Journal of immunology (Baltimore, Md.: 1950) 149: 1666-1670.
72. Pinsky, M. R., J. L. Vincent, J. Deviere, M. Alegre, R. J. Kahn, and E. Dupont. 1993. Serum cytokine levels in human septic shock. Relation to multiple-system organ failure and mortality. Chest 103: 565-575.
73. Hayworth, J. L., K. J. Kasper, M. Leon-Ponte, C. A. Herfst, D. Yue, W. C. Brintnell, D. M. Mazzuca, D. E. Heinrichs, E. Cairns, J. Madrenas, D. W. Hoskin, J. K. McCormick, and S. M. Haeryfar. 2009. Attenuation of massive cytokine response to the staphylococcal enterotoxin B superantigen by the innate immunomodulatory protein lactoferrin. Clinical and experimental immunology 157: 60-70.
74. Matsukawa, A., C. M. Hogaboam, N. W. Lukacs, P. M. Lincoln, H. L. Evanoff, R. M. Strieter, and S. L. Kunkel. 2000. Expression and contribution of endogenous IL-13 in an experimental model of sepsis. Journal of immunology (Baltimore, Md.: 1950) 164: 2738-2744.
75. Wynn, T. A. 2003. IL-13 effector functions. Annual review of immunology 21: 425-456.
76. Muchamuel, T., S. Menon, P. Pisacane, M. C. Howard, and D. A. Cockayne. 1997. IL-13 protects mice from lipopolysaccharide-induced lethal endotoxemia: correlation with down-modulation of TNF-alpha, IFN-gamma, and IL-12 production. Journal of immunology (Baltimore, Md.: 1950) 158: 2898-2903.
77. Yu, K. O., and S. A. Porcelli. 2005. The diverse functions of CD1d-restricted NKT cells and their potential for immunotherapy. Immunology letters 100: 42-55.
78. Cannaday, J. E. 1948. Some uses of undetached omentum in surgery. American journal of surgery 76: 502-505.
79. Van Vugt, E., E. A. Van Rijthoven, E. W. Kamperdijk, and R. H. Beelen. 1996. Omental milky spots in the local immune response in the peritoneal cavity of rats. The Anatomical record 244: 235-245.
80. Lynch, L., D. O'Shea, D. C. Winter, J. Geoghegan, D. G. Doherty, and C. O'Farrelly. 2009. Invariant NKT cells and CD1d(+) cells amass in human omentum and are depleted in patients with cancer and obesity. European journal of immunology 39: 1893-1901.
81. Carlow, D. A., M. R. Gold, and H. J. Ziltener. 2009. Lymphocytes in the peritoneum home to the omentum and are activated by resident dendritic cells. Journal of immunology (Baltimore, Md.: 1950) 183: 1155-1165.
82. Lee, H. H., E. H. Meyer, S. Goya, M. Pichavant, H. Y. Kim, X. Bu, S. E. Umetsu, J. C. Jones, P. B. Savage, Y. Iwakura, J. M. Casasnovas, G. Kaplan, G. J. Freeman, R. H. DeKruyff, and D. T. Umetsu. 2010. Apoptotic cells activate NKT cells through T cell Ig-like mucin-like-1 resulting in airway hyperreactivity. Journal of immunology (Baltimore, Md.: 1950) 185: 5225-5235.
83. Wermeling, F., S. M. Lind, E. D. Jordo, S. L. Cardell, and M. C. Karlsson. 2010. Invariant NKT cells limit activation of autoreactive CD1d-positive B cells. The Journal of experimental medicine 207: 943-952.
84. Hotchkiss, R. S., G. Monneret, and D. Payen. 2013. Sepsis-induced immunosuppression: from cellular dysfunctions to immunotherapy. Nature reviews. Immunology 13: 862-874.
85. Fadok, V. A., D. L. Bratton, A. Konowal, P. W. Freed, J. Y. Westcott, and P. M. Henson. 1998. Macrophages that have ingested apoptotic cells in vitro inhibit proinflammatory cytokine production through autocrine/paracrine mechanisms involving TGF-beta, PGE2, and PAF. The Journal of clinical investigation 101: 890-898.
86. Heffernan, D. S., S. F. Monaghan, C. S. Chung, W. G. Cioffi, S. Gravenstein, and A. Ayala. 2013. A divergent response of innate regulatory T-cells to sepsis in humans: Circulating invariant natural killer T-cells are preserved. Human immunology.
87. Cohen, J., S. Opal, and T. Calandra. 2012. Sepsis studies need new direction. The Lancet Infectious Diseases 12: 503-505.
88. Vincent, J. L., Q. Sun, and M. J. Dubois. 2002. Clinical trials of immunomodulatory therapies in severe sepsis and septic shock. Clinical infectious diseases: an official publication of the Infectious Diseases Society of America 34: 1084-1093.
89. Meisel, C., J. C. Schefold, R. Pschowski, T. Baumann, K. Hetzger, J. Gregor, S. Weber-Carstens, D. Hasper, D. Keh, H. Zuckermann, P. Reinke, and H. D. Volk. 2009. Granulocyte-macrophage colony-stimulating factor to reverse sepsis-associated immunosuppression: a double-blind, randomized, placebo-controlled multicenter trial. American journal of respiratory and critical care medicine 180: 640-648.
90. Hall, M., N. Knatz, C. Vetterly, S. Tomarello, M. Wewers, H. Volk, and J. Carcillo. 2011. Immunoparalysis and nosocomial infection in children with multiple organ dysfunction syndrome. Intensive care medicine 37: 525-532.
91. Morre, M., and S. Beq. 2012. Interleukin-7 and immune reconstitution in cancer patients: a new paradigm for dramatically increasing overall survival. Targ Oncol 7: 55-68.
92. Mackall, C. L., T. J. Fry, and R. E. Gress. 2011. Harnessing the biology of IL-7 for therapeutic application. Nature reviews. Immunology 11: 330-342.
93. Rosenberg, S. A., C. Sportes, M. Ahmadzadeh, T. J. Fry, L. T. Ngo, S. L. Schwarz, M. Stetler-Stevenson, K. E. Morton, S. A. Mavroukakis, M. Morre, R. Buffet, C. L. Mackall, and R. E. Gress. 2006. IL-7 administration to humans leads to expansion of CD8+ and CD4+ cells but a relative decrease of CD4+ T-regulatory cells. Journal of immunotherapy (Hagerstown, Md.: 1997) 29: 313-319.
94. Pellegrini, M., T. Calzascia, J. G. Toe, S. P. Preston, A. E. Lin, A. R. Elford, A. Shahinian, P. A. Lang, K. S. Lang, M. Morre, B. Assouline, K. Lahl, T. Sparwasser, T. F. Tedder, J. H. Paik, R. A. DePinho, S. Basta, P. S. Ohashi, and T. W. Mak. 2011. IL-7 engages multiple mechanisms to overcome chronic viral infection and limit organ pathology. Cell 144: 601-613.
95. Kasten, K. R., P. S. Prakash, J. Unsinger, H. S. Goetzman, L. G. England, C. M. Cave, A. P. Seitz, C. N. Mazuski, T. T. Zhou, M. Morre, R. S. Hotchkiss, D. A. Hildeman, and C. C. Caldwell. 2010. Interleukin-7 (IL-7) treatment accelerates neutrophil recruitment through gamma delta T-cell IL-17 production in a murine model of sepsis. Infection and immunity 78: 4714-4722.

Through the embodiments that are illustrated and described, the currently contemplated best mode of making and using the invention is described.

Without further elaboration, it is believed that one of ordinary skill in the art can, based on the description presented herein, utilize the present invention to the full extent. All publications cited are incorporated by reference.

1. A method of treating sepsis in a subject comprising administering to the subject a Th2-polarizing iNKT cell agonist or administering to the subject a Th2 phenotype modulated iNKT cell.

2. The method of claim 1, wherein the Th2-polarizing iNKT cell agonist is a glycolipid ligand of iNKT cells.

3. The method of claim 1, wherein the Th2-polarizing iNKT cell agonist is α-Galactosyl Ceramide or an α-Galactosyl Ceramide analog or derivative.

4. The method of claim 1, wherein the Th2-polarizing iNKT cell agonist is synthetic glycolipid OCH.

5. The method of claim 1, wherein the Th2-polarizing iNKT cell agonist is C20:2.

6. (canceled)

7. The method of claim 1, wherein sepsis is intra-abdominal sepsis.

8-14. (canceled)

15. A method of reducing apoptosis in a subject comprising administering to the subject a Th2-polarizing iNKT cell agonist or administering to the subject a Th2 phenotype modulated iNKT cell.

16. The method of claim 15, wherein the Th2-polarizing iNKT cell agonist is a glycolipid ligand of iNKT cells.

17. The method of claim 15, wherein the Th2-polarizing iNKT cell agonist is α-Galactosyl Ceramide or an α-Galactosyl Ceramide analog or derivative.

18. The method of claim 15, wherein the Th2-polarizing iNKT cell agonist is synthetic glycolipid OCH.

19. The method of claim 15, wherein the Th2-polarizing iNKT cell agonist is C20:2.

20. (canceled)

21. The method of claim 15, wherein the method reduces apoptosis in splenic B and T lymphocytes and macrophages.

22-28. (canceled)

29. A method of detecting early stage sepsis in a subject in need thereof, the method comprising (a) measuring a test ratio of circulating iNKT cells relative to the total circulating T cells in the subject, and (b) comparing the test ratio to a control ratio of subjects without sepsis, wherein a test ratio higher than the control ratio is indicative of early stage sepsis.

30-43. (canceled)

44. The method of claim 16, wherein the apoptosis is sepsis-induced apoptosis.
